(19) United States
(12) Patent Application Publication
(10) Pub. No.: US 2025/0177538 A1
(43) Pub. Date: Jun. 5, 2025

(54) Methods for synthesizing deuterated formylindoles

(71) Applicant: ARIZONA BOARD OF REGENTS ON BEHALF OF THE UNIVERSITY OF ARIZONA, Tucson, AZ (US)

(72) Inventor: Wei Wang, Tucson, AZ (US)

(21) Appl. No.: 18/877,162

(22) PCT Filed: Jun. 21, 2023
(86) PCT No.: PCT/US2023/068774

§ 371 Date: Dec. 19, 2024

Related U.S. Application Data
(60) Provisional application No. 63/354,191, filed on Jun. 21, 2022.

(51) Int. Cl. A61K 47/54

(52) U.S. Cl. CPC A61K 47/545; A61K 47/542

(57) ABSTRACT

Exemplary materials, methods and techniques disclosed and contemplated herein generally relate to the photochemical synthesis of various deuterated 3-formyl indoles using deuterated glyoxylic acid (CDOCO2D).

### CROSS-REFERENCE TO RELATED APPLICATION(S)

This application claims priority to U.S. Provisional Patent Application No. 63/354,191, filed on Jun. 21, 2022, the entire contents of which are incorporated herein by reference.

### STATEMENT OF GOVERNMENT INTEREST

This invention was made with government support under Grant No. GM125920 awarded by the National Institutes of Health. The government has certain rights in the invention.

### TECHNICAL FIELD

Exemplary materials, methods and techniques disclosed and contemplated herein generally relate to the photochemical synthesis of various deuterated 3-formyl indoles using deuterated glyoxylic acid (CDOCO₂D).

### INTRODUCTION

The recent surge in application of deuterium in pharmaceuticals, including the FDA approval of the first deuterated drug, Austedo® in 2017, demands practical, cost-effective methods for the synthesis of versatile deuterated functionalities and structures. Indoles are the ‘privileged’ core structure featured in numerous natural products and medicinally relevant molecules. What is needed are new methods for preparing deuterated molecules, particularly molecules with a privileged core structure, such as indoles.

### SUMMARY

In one aspect, the present disclosure provides a method of preparing deuterated compound of formula (I), or a salt thereof,

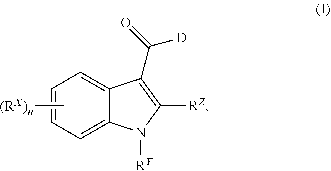

wherein:
R^{X} is halogen, C₁₋₄alkyl, C₁₋₂haloalkyl, R^{a}, —NO₂, —CN, —CO₂C₁₋₄alkyl, —C(O)C₁₋₄alkyl, —C(O)H, —OC₁₋₃alkylene-R^{a}, —C₁₋₃alkylene-R^{a}, -L¹-A¹, or -L²-A²;
R^{a} is C₃₋₄cycloalkyl, —OC₁₋₄alkyl, —OC₁₋₂haloalkyl, —OC₃₋₄cycloalkyl, or phenyl, wherein, at each occurrence, the phenyl is optionally substituted with 1-2 substituents selected from the group consisting of halogen, C₁₋₄alkyl, C₁₋₂haloalkyl, —NO₂, —CN, —OC₁₋₄alkyl, —OC₁₋₂haloalkyl, or —OC₃₋₄cycloalkyl;
R^{Y} is hydrogen, C₁₋₄alkyl, or an amine protecting group;
R^{Z} is hydrogen, C₁₋₄alkyl, C₂₋₄alkenyl, or unsubstituted phenyl;
L¹ is —C₁₋₃alkylene- or —OC₁₋₃alkylene-;
A¹ is a drug moiety;
   wherein A¹ is linked to L¹ by

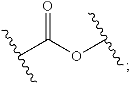 [SMILES: *OC(*)=O]

L² is —NH—;
A² is a drug moiety;
   wherein A² is linked to L² by

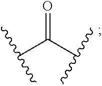 [SMILES: *C(*)=O]

and
n is 0, 1, or 2;
the method comprising:
   mixing D-CO₂D with a compound of formula (II),

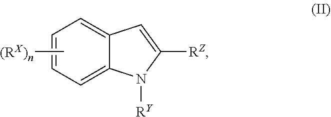

in an organic solvent to form a mixture; and
exposing the mixture of (i) to light, thereby producing the deuterated compound of formula (I), or a salt thereof.

In various instances, exposing the mixture of (i) to light may occur in the presence of air. In some instances, the mixture may be essentially water (H₂O) free. In some instances, the base may be sodium acetate (NaOAc). In some instances, the mixture may be catalyst free. In some instances, the solvent may be a polar aprotic solvent.

In another aspect, the disclosure provides a deuterated compound of formula (I), or a salt thereof,

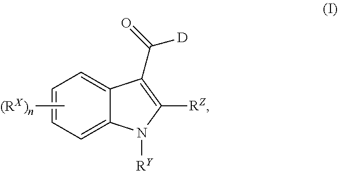

wherein:
R^{X} is halogen, C₁₋₄alkyl, C₁₋₂haloalkyl, R^{a}, —NO₂, —CN, —CO₂C₁₋₄alkyl, —C(O)C₁₋₄alkyl, —C(O)H, —OC₁₋₃alkylene-R^{a}, —C₁₋₃alkylene-R^{a}, -L¹-A¹, or L²-A²;
R^{a} is C₃₋₄cycloalkyl, —OC₁₋₄alkyl, —OC₁₋₂haloalkyl, —OC₃₋₄cycloalkyl, or phenyl, wherein, at each occurrence, the phenyl is optionally substituted with 1-2 substituents selected from the group consisting of halogen, C₁₋₄alkyl, C₁₋₂haloalkyl, —NO₂, —CN, —OC₁₋₄alkyl, —OC₁₋₂haloalkyl, or —OC₃₋₄cycloalkyl;
R^{Y} is hydrogen, C₁₋₄alkyl, or an amine protecting group;
R^{Z} is hydrogen, C₁₋₄alkyl, C₂₋₄alkenyl, or unsubstituted phenyl;
L¹ is —C₁₋₃alkylene- or —OC₁₋₃alkylene-;
A¹ is a drug moiety;
   wherein A¹ is linked to L¹ by

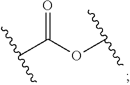 [SMILES: *OC(*)=O]

L² is —NH—;
A² is a drug moiety;
   wherein A² is linked to L² by:

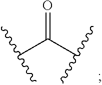 [SMILES: *C(*)=O]

and
n is 0, 1, or 2.

In some instances, n may be 0 or 1. In some instances, R^{X} may be halogen, C₁₋₄alkyl, —NO₂, —CN, —OC₁₋₄alkyl, —CO₂C₁₋₄alkyl, —C(O)H, -L¹-A¹, or -L²-A². In some instances, L¹ may be —CH₂—. In some instances, the drug moiety at A¹ or A² may be a non-steroidal anti-inflammatory drug (NSAID) moiety or a β-lactamase inhibitor moiety. In some instances, R^{X} may be selected from the group consisting of:

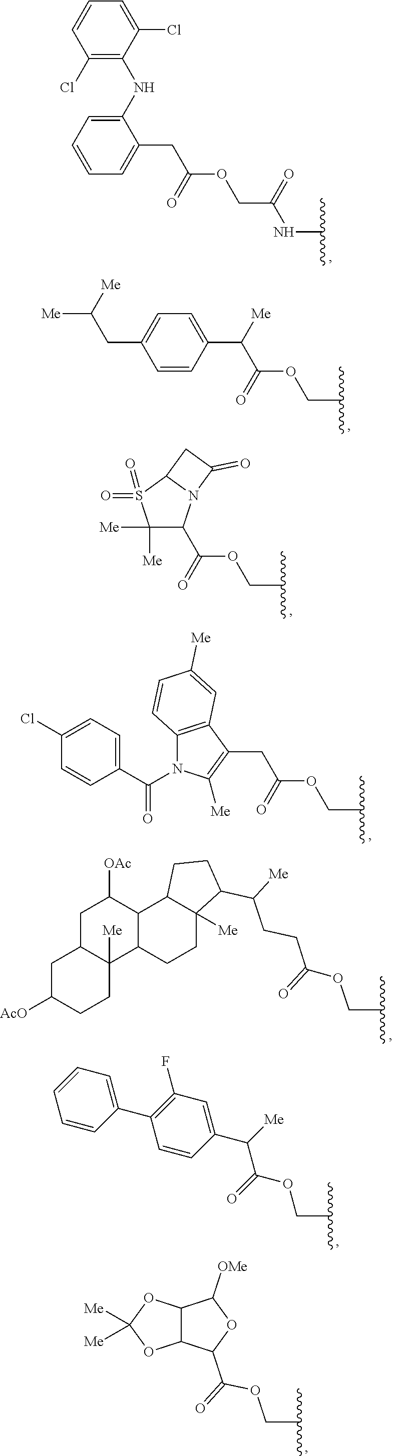

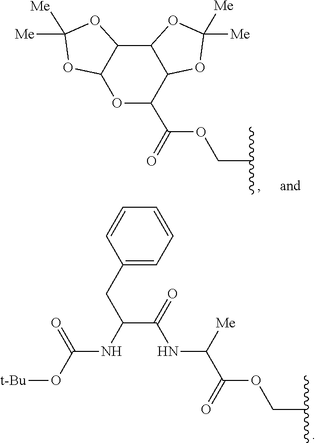

In some instances, R^{Y} may be hydrogen, C₁₋₄alkyl,

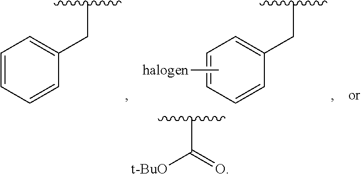

In some instances, R^{Z} may be hydrogen, methyl,

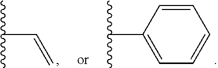

In some instances, the compound may be selected from the group consisting of:

[chemical structure]

In some instances, the compound may have at least 50% deuterium incorporation at the deuterium label. In some instances, the compound may have at least 75% deuterium incorporation at the deuterium label. In some instances, the compound may have at least 90% deuterium incorporation at the deuterium label. In some instances, the compound may have at least 95% deuterium incorporation at the deuterium label. In some instances, the compound may have at least 99% deuterium incorporation at the deuterium label.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### BRIEF DESCRIPTION OF THE DRAWINGS

FIG. 1 shows various exemplary indole starting materials.

FIG. 2 shows exemplary 3-formyl indoles prepared via catalyst free photochemical 3-formylation of indoles with glyoxylic acid.

FIGS. 3A-3B shows the scope of deuterated 3-formyl indoles prepared via catalyst free photochemical 3-formylation of indoles with deuterated glyoxylic acid (CDOCO₂D).

FIG. 3A schematically shows an exemplary synthetic method for preparing various deuterated 3-formyl indoles and example products 3-a-d through 3-v-d.

FIG. 3B shows exemplary deuterated 3-formyl indole products having a drug moiety attached (example products 3-w-d through 3-ae-d).

FIG. 4A is a cyclic voltammogram for 1-methyl-1H-indole-3-carbaldehyde (recorded at a scan rate of 0.2 V/s).

FIG. 4B is a cyclic voltammogram for 2-oxo-2-phenylacetic acid (recorded at a scan rate of 0.2 V/s).

FIG. 4C is a cyclic voltammogram for 1H-indole (recorded at a scan rate of 0.2 V/s).

FIG. 4D is a cyclic voltammogram] for 1-methyl-1H-indole (recorded at a scan rate of 0.2 V/s).

FIG. 4E is a cyclic voltammogram for sodium 2-hydroxy-2-(1-methyl-1H-indol-3-yl)acetate (recorded at a scan rate of 0.2 V/s).

FIG. 4F is a cyclic voltammogram for indoline-2,3-dione (recorded at a scan rate of 0.2 V/s).

FIG. 4G is a blank (control) cyclic voltammogram recorded at a scan rate of 0.2 V/s.

FIG. 5 graphically illustrates the reaction yield over time for the reaction between N-methyl indole and glyoxylic acid in dark conditions or under nitrogen atmosphere (N₂) conditions.

FIG. 6 shows ultraviolet-visible (UV-vis) spectra of sodium 2-hydroxy-2-(1-methyl-1H-indol-3-yl)acetate (hydroxy acid intermediate 5a), isatin, 6-C1-isatin, N-methyl-3-formylindole, and N-methyl indole.

FIG. 7 schematically illustrates the proposed catalytic cycle based on the mechanistic studies.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various way.

### I. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

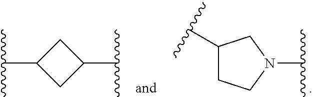 [SMILES: *C1CC(*)C1 ; *C1CCN(*)C1]

Cycloalkylene and heterocyclylene include a geminal divalent groups such as 1,1-C₃₋₆cycloalkylene (i.e.,

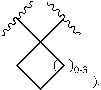

A further example is 1,1-cyclopropylene (i.e.,

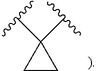 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl(thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

In the compounds of formula (I), and any subformulas, any “hydrogen” or “H,” whether explicitly recited or implicit in the structure, encompasses hydrogen isotopes ¹H (protium) and ²H (deuterium).

The present disclosure also includes an isotopically-labeled compound (e.g., deuterium labeled), where an atom in the isotopically-labeled compound is specified as a particular isotope of the atom. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²p, ³⁵S, ¹⁸F, and ³⁶Cl, respectively.

The term “isotopically enriched,” as used herein with reference to any particular isotope of any particular atom of a compound, means that in a composition comprising a plurality of molecules of the compound, the amount (e.g., fraction, ration or percentage) of the plurality of molecules having the particular isotope at the particular atom is substantially greater than the natural abundance of the particular isotope, due to synthetic enrichment of the particular atom with the particular isotope.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically labeled atom (e.g., % deuterium incorporation at a deuterium label).

For example, a compound with an isotopically enriched deuterium (²H, denoted as “D”) atom at one or more particular locations includes a plurality of molecules of the compound, where as a result of synthetic enrichment, the percentage of the plurality of molecules having deuterium at each of the one or more particular locations is greater than about 1% (the natural abundance of deuterium is substantially less than 1%), and in many cases is substantially greater than about 1%.

In some cases, a compound with an isotope incorporated at a particular isotopically labeled atom may include a plurality of molecules of the compound, where the amount of the plurality of molecules having the isotope at the particular isotopically labeled atom may be at least about two-or-more-fold greater than the natural abundance of the isotope at that atom, including but not limited to at least about two-fold, at least about three-fold, at least about four-fold, at least about five-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, and at least about 200-fold, among others. In some cases, a compound with an isotope at a particular isotopically labeled atom also may include a plurality of molecules of the compound where, as a result of synthetic enrichment, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 99.5% of the plurality of molecules have the isotope at the isotopically labeled atom.

The term “natural abundance,” as used herein with reference to any particular isotope of an element, refers to the abundance of the isotope as naturally found on the planet Earth. For example, the natural abundance of ⁵N on the planet Earth is generally regarded to be about 0.37% (i.e., substantially less than about 1%), while the natural abundance of deuterium (²H) on the planet Earth is generally regarded to be about 0.015% (i.e., substantially less than about 1%).

The term “photochemical,” means relating to or caused by the chemical action of light.

### II. Exemplary Compounds

The present disclosure relates to deuterated 3-formyl indoles and methods for synthesizing the same.

### A. Deuterated Compounds of Formula (I)

In one aspect, the present disclosure provides deuterated compounds of formula (I),

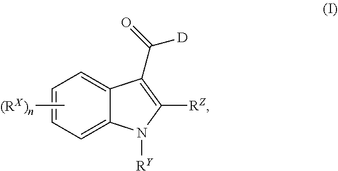

or a salt thereof, wherein R^{X}, R^{Y}, R^{Z}, and n are as defined here.

In the following, various embodiments of the compounds of formula (I) are disclosed. The first embodiment is denoted E1, the second embodiment is denoted E2 and so forth.

E1. A compound of formula (I), or a pharmaceutically acceptable salt thereof,

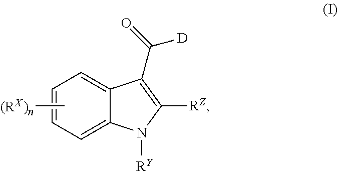

wherein:
R^{X} is halogen, C₁₋₄alkyl, C₁₋₂haloalkyl, R^{a}, —NO₂, —CN, —CO₂C₁₋₄alkyl, —C(O)C₁₋₄alkyl, —C(O)H, —OC₁₋₃alkylene-R^{a}, —C₁₋₃alkylene-R^{a}, -L¹-A¹, or -L²-A²;
R^{a} is C₃₋₄cycloalkyl, —OC₁₋₄alkyl, —OC₁₋₂haloalkyl, —OC₃₋₄cycloalkyl, or phenyl, wherein, at each occurrence, the phenyl is optionally substituted with 1-2 substituents selected from the group consisting of halogen, C₁₋₄alkyl, C₁₋₂haloalkyl, —NO₂, —CN, —OC₁₋₄alkyl, —OC₁₋₂haloalkyl, or —OC₃₋₄cycloalkyl;
R^{Y} is hydrogen, C₁₋₄alkyl, or an amine protecting group;
R^{Z} is hydrogen, C₁₋₄alkyl, C₂₋₄alkenyl, or unsubstituted phenyl;
L¹ is —C₁₋₃alkylene- or —OC₁₋₃alkylene-;
A¹ is a drug moiety;
   wherein A¹ is linked to L¹ by

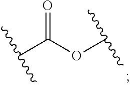 [SMILES: *OC(*)=O]

L² is —NH—;
A² is a drug moiety;
   wherein A² is linked to L² by:

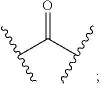 [SMILES: *C(*)=O]

and
n is 0, 1, or 2.

E2. The compound of E1, or a salt thereof, wherein n is 0 or 1.

E3. The compound of E1 or E2, or a salt thereof, wherein R^{X} is halogen, C₁₋₄alkyl, —NO₂, —CN, —OC₁₋₄alkyl, —CO₂C₁₋₄alkyl, —C(O)H, -L¹-A¹, or -L²-A².

E4. The compound of any one of E1-E3, or a salt thereof, wherein L¹ is —CH₂—.

E5. The compound of any one of E1-E4, or a salt thereof, wherein the drug moiety at A¹ or A² is a non-steroidal anti-inflammatory drug (NSAID) moiety or a R-lactamase inhibitor moiety.

E6. The compound of any one of E1-E5, or a salt thereof, wherein R^{X} is selected from the group consisting of:

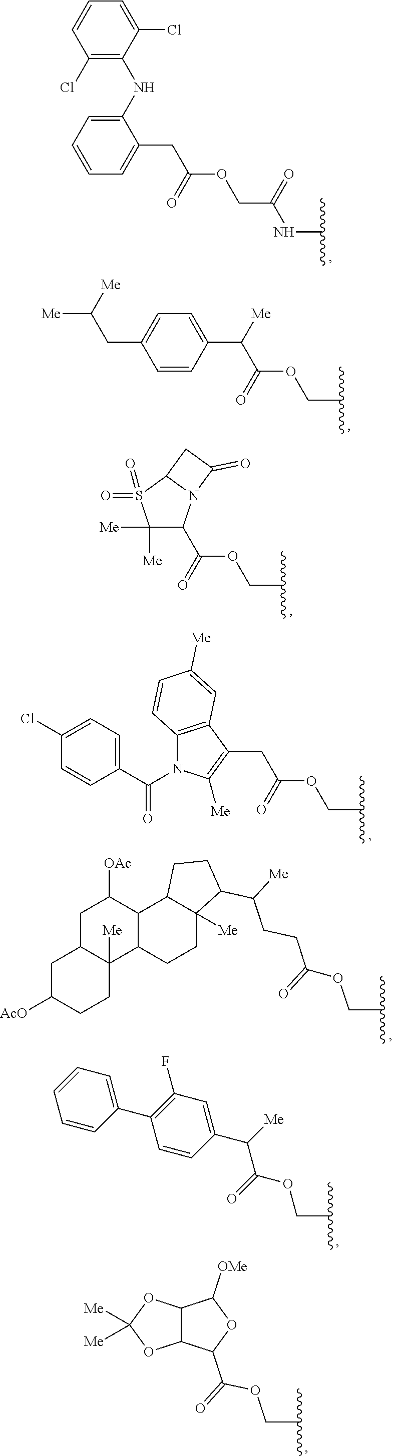

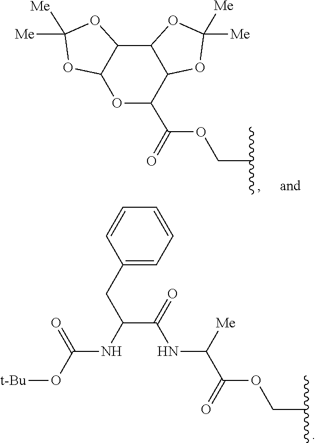

E7. The compound of any one of E1-E6, or a salt thereof, wherein R^{Y} is hydrogen, C₁₋₄alkyl,

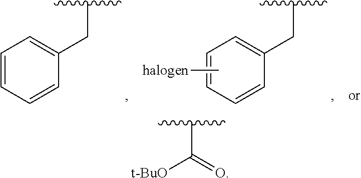

E8. The compound of any one of E1-E7, or a salt thereof, wherein R^{Z} is hydrogen, methyl,

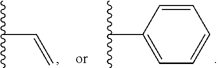

E9. The compound of any one of E1-E8, selected from the group consisting of:

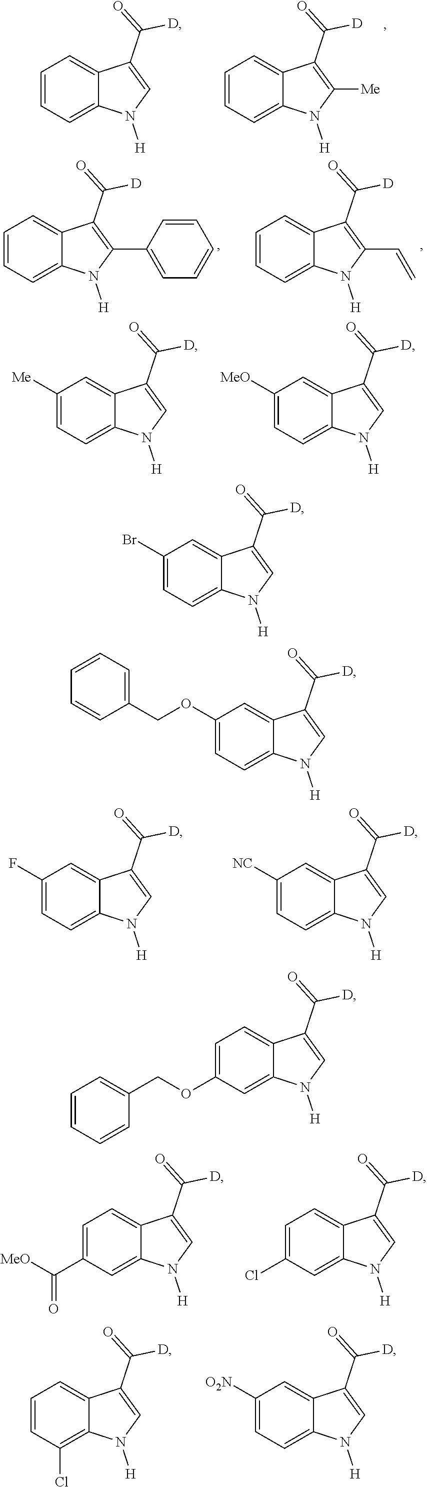

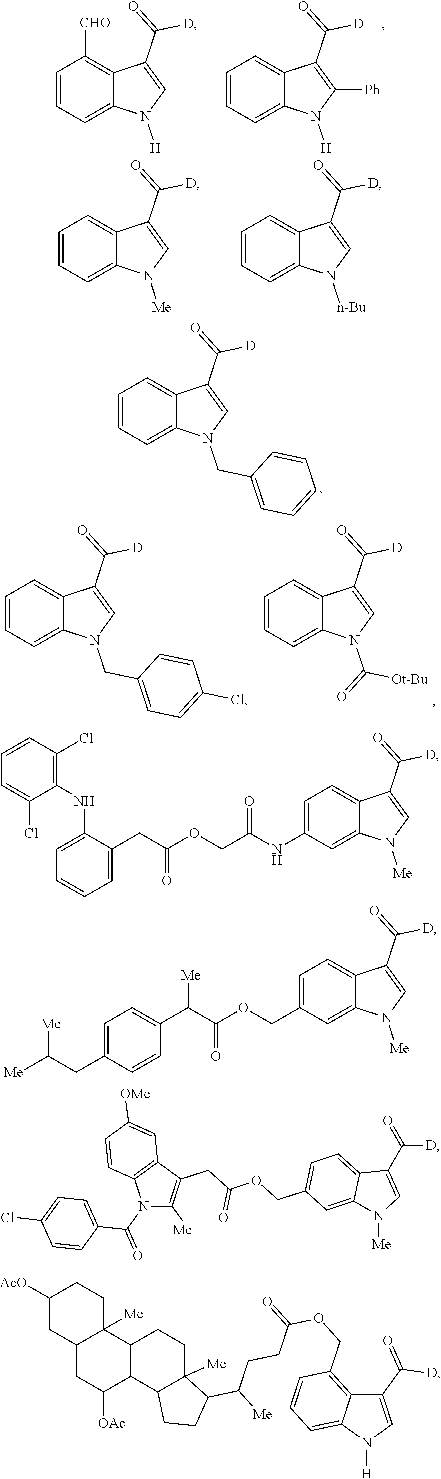

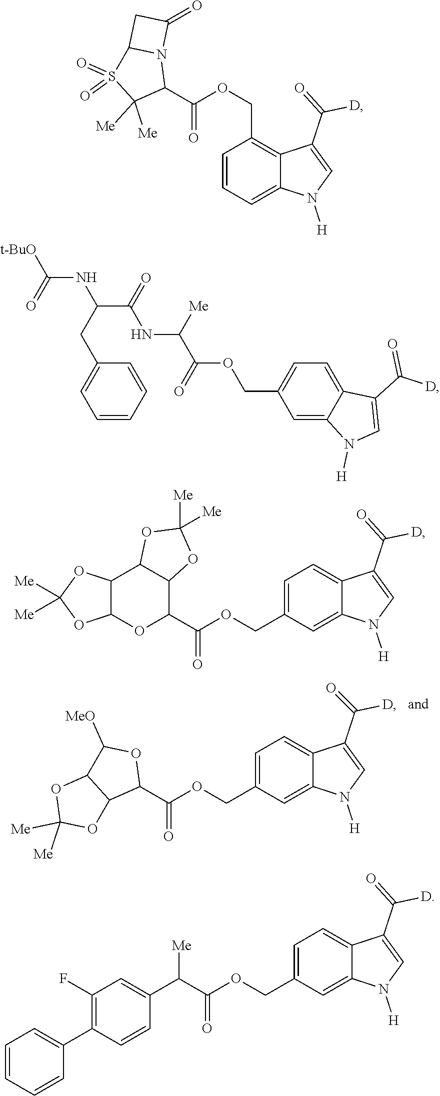

E10. The compound of any one of E1-E9, or a salt thereof, wherein that compound has at least 50% deuterium incorporation at the deuterium label.

E11. The compound of any one of E1-E10, or a salt thereof, wherein that compound has at least 75% deuterium incorporation at the deuterium label.

E12. The compound of any one of E1-E11, or a salt thereof, wherein that compound has at least 90% deuterium incorporation at the deuterium label.

E13. The compound of any one of E1-E12, or a salt thereof, wherein that compound has at least 95% deuterium incorporation at the deuterium label.

E14. The compound of any one of E1-E13, or a salt thereof, wherein that compound has at least 99% deuterium incorporation at the deuterium label.

### B. Compounds of Formula (II)

In another aspect, the present disclosure provides compounds of formula (II),

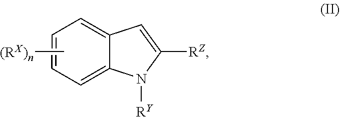

or a salt thereof, wherein R^{X}, R^{Y}, R^{Z}, and n are as defined herein. In various instances, exemplary compounds of formula (II) may be reacted with deuterated glyoxylic acid (CDOCO₂D) to provide deuterated compounds of formula (I).

### III. Exemplary Synthetic Methods

Deuterated compounds of formula (I) may be prepared by exemplary synthetic processes depicted in the following schemes.

Abbreviations which have been used in the descriptions and schemes that follow are:
CFL is compact fluorescent light;
W is watts;
LED is light emitting diode,
MeCN is acetonitrile;
THF is tetrahydrofuran;
DMSO is dimethylsulfoxide;
DCM is dichloromethane;
DMF is N,N-dimethylformamide;
NaOAc is sodium acetate;
KOAc is potassium acetate;
LiOAc is lithium acetate;
PC is photocatalyst;
eq, eq., or equiv is equivalent(s); and
Molar is M.

General Scheme 1, below, illustrates the method for preparing deuterated compounds of formula (I), described herein.

As shown in General Scheme 1 below, deuterated compounds of formula (I) may be prepared by mixing D-CO₂D with a compound of formula (II) in a solvent to form a mixture, and exposing the mixture to light.

### General Scheme 1

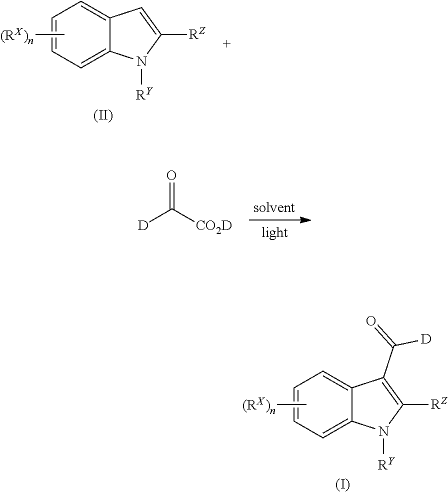

### A. Exemplary Mixtures

### 1. Molar Ratios of Deuterated Glyoxylic Acid to Compound of Formula (II)

For exemplary mixtures, in various instances, the molar ratio of deuterated glyoxylic acid (CDOCO₂D) to compound of formula (II) may be 2.0 to 4.0. In some instances, the molar ratio of deuterated glyoxylic acid (CDOCO₂D) to compound of formula (II) may be 2.1 to 3.9; 2.2 to 3.8; 2.3 to 3.7; 2.4 to 3.6; 2.5 to 3.5; 2.6 to 3.4; 2.7 to 3.3; 2.8 to 3.2; or 2.9 to 3.1. In some instances, the molar ratio of deuterated glyoxylic acid (CDOCO₂D) to compound of formula (II) may be no greater than 4.0; no greater than 3.9; no greater than 3.8; no greater than 3.7; no greater than 3.6; no greater than 3.5; no greater than 3.4; no greater than 3.3; no greater than 3.2; no greater than 3.1; no greater than 3.0; no greater than 2.9; no greater than 2.8; no greater than 2.7; no greater than 2.6; no greater than 2.5; no greater than 2.4; no greater than 2.3; no greater than 2.2; or no greater than 2.1. In some instances, the molar ratio of deuterated glyoxylic acid (CDOCO₂D) to compound of formula (II) may be no less than 2.0; no less than 2.1; no less than 2.2; no less than 2.3; no less than 2.4; no less than 2.5; no less than 2.6; no less than 2.7; no less than 2.8; no less than 2.9; no less than 3.0; no less than 3.1; no less than 3.2; no less than 3.3; no less than 3.4; no less than 3.5; no less than 3.6; no less than 3.7; no less than 3.8; or no less than 3.9.

### 2. Solvents

In various instances, the solvent of the mixture may be an organic solvent. In some instances, the organic solvent may be a polar aprotic solvent. Exemplary polar aprotic solvents include, without limitation, acetonitrile (MeCN), tetrahydrofuran (THF), dichloromethane (DCM), 1,4-dioxane, dimethylsulfoxide (DMSO), N,N-dimethylformamide (DMF), and combinations thereof.

In various instances, exemplary mixtures may be essentially water free. As used herein, “essentially water free” means that no water is present, or water is present in a nominal amount.

### 3. Compound of Formula (II) Concentrations

In various instances, the compound of formula (II) may be present in the mixture at a concentration of 0.04 M to 0.16 M. In some instances, the concentration of the compound of formula (II) in the solvent may be 0.05 M to 0.15 M; 0.06 to 0.14 M; 0.07 M to 0.13 M; 0.08 M to 0.12 M; or 0.09 M to 0.11 M. In some instances, the compound of formula (II) may be present in the mixture at a concentration of no greater than 0.16 M; no greater than 0.15 M; no greater than 0.14 M; no greater than 0.13 M; no greater than 0.12 M; no greater than 0.11 M; no greater than 0.10 M; no greater than 0.09 M; no greater than 0.08 M; no greater than 0.07 M; or no greater than 0.06 M. In some instances, the compound of formula (II) may be present in the mixture at a concentration of no less than 0.04 M; no less than 0.05 M; no less than 0.06 M; no less than 0.07 M; no less than 0.08 M; no less than 0.09 M; no less than 0.1 M; no less than 0.11 M; no less than 0.12 M; no less than 0.13 M; or no less than 0.14 M.

### 4. Photocatalysts

In various instances, exemplary mixtures may be catalyst free, e.g., photocatalyst (PC) free. In other instances, the mixture may comprise a catalyst. The catalyst may be a photocatalyst. Exemplary photocatalysts include, without limitation, 6-C1-isatin, isatin, Eosin Y, Rose Bengal, and combinations thereof.

When the photocatalyst is present, exemplary mixtures may comprise the photocatalyst at 5 mole % (mol %) to 15 mol %. In some instances, exemplary mixtures may comprise the photocatalyst, when present, at 6 mol % to 14 mol %; 7 mol % to 13 mol %; 8 mol % to 12 mol %; or 9 mol % to 11 mol %. In some instances, exemplary mixtures may comprise the photocatalyst, when present, at no greater than 15 mol %; no greater than 14 mol %; no greater than 13 mol %; no greater than 12 mol %; no greater than 11 mol %; no greater than 10 mol %; no greater tan 9 mol %; no greater than 8 mol %; no greater than 7 mol %; or no greater than 6 mol %. In some instances, exemplary mixtures may comprise the photocatalyst, when present, at no less than 5 mol %; no less than 6 mol %; no less than 7 mol %; no less than 8 mol %; no less than 9 mol %; no less than 10 mol %; no less than 11 mol %; no less than 12 mol %; no less than 13 mol %; or no less than 14 mol %.

### 5. Bases

In various instances, exemplary mixtures may further comprise a base. Exemplary bases include, without limitation, organic bases, inorganic bases, and combinations thereof. In various instances, the base may comprise an electron-accepting cation (e.g., sodium (Na⁺), lithium (Li⁺), and/or potassium (K⁺)) and an electron-donating anion (e.g., acetate (OAc⁻), carbonate (C₃⁻²)). Example bases that may be present in the mixture include, without limitation, sodium acetate (NaOAc), potassium acetate (KOAc), lithium acetate (LiOAc), and combinations thereof.

When a base is present in the mixture, the molar ratio of base to compound of formula (II) may be 1.0 to 3.0. In some instances, when a base is present in the mixture, the molar ratio of base to compound of formula (II) is 1.1 to 2.9; 1.2 to 2.8; 1.3 to 2.7 to 1.4 to 2.6; 1.5 to 2.5; 1.6 to 2.4; 1.7 to 2.3; 1.8 to 2.2; or 1.9 to 2.1. In some instances, when a base is present in the mixture, the molar ratio of base to compound of formula (II) is no greater than 2.9; no greater than 2.8; no greater than 2.7; no greater than 2.6; no greater than 2.5; no greater than 2.4; no greater than 2.3; no greater than 2.2; no greater than 2.1; no greater than 2.0; no greater than 1.9; no greater than 1.8; no greater than 1.7; no greater than 1.6; no greater than 1.5; no greater than 1.4; no greater than 1.3; no greater than 1.2; or no greater than 1.1. In some instances, when a base is present in the mixture, the molar ratio of base to compound of formula (II) is no less 1.0; no less than 1.1; no less than 1.2; no less than 1.3; no less than 1.4; no less than 1.5; no less than 1.6; no less than 1.7; no less than 1.8; no less than 1.9; no less than 2.0; no less than 2.1; no less than 2.2; no less than 2.3; no less than 2.4; no less 2.5; no less than 2.6; no less than 2.7; no less than 2.8; or no less than 2.9.

### B. Exemplary Light Exposure Conditions

### 1. Light Source

In some instances, the light may be provided from one or more compact fluorescent light (CFL) sources, e.g., 2×42 W CFL. In other instances, the light may be provided from one or more light emitting diode (LED) light sources. Exemplary LED light sources include, without limitation, white LED, purple LED, blue LED, and combinations thereof.

### 2. Atmosphere

Exposing the mixture to light may occur in the presence of air. In other instances, exposing the mixture to light may occur in the absence of oxygen (O₂). Exposing the mixture to light may occur under argon (Ar) atmosphere or nitrogen (N₂) atmosphere.

### 3. Time Period

In various instances, exposing the mixture to light may occur for a time period of 12 to 48 hours. In some instances, exposing the mixture to light may occur for a time period of 14 to 46 hours; 15 to 45 hours; 18 to 42 hours; 20 to 40 hours; 22 to 38 hours; 23 to 37 hours; 24 to 36 hours; 25 to 35 hours; 26 to 34 hours; 27 to 33 hours; or 28 to 32 hours. In some instances, exposing the mixture to light may occur for a time period of no greater than 48 hours; no greater than 45 hours; no greater than 42 hours; no greater than 40 hours; no greater than 38 hours; no greater than 36 hours; no greater than 32 hours; no greater than 30 hours; no greater than 28 hours; no greater than 26 hours; no greater than 24 hours; no greater than 20 hours; no greater than 18 hours; no greater than 16 hours; or no greater than 14 hours. In some instances, exposing the mixture to light may occur for a time period of no less than 12 hours; no less than 14 hours; no less than 16 hours; no less than 18 hours; no less than 20 hours; no less than 22 hours; no less than 24 hours; no less than 26 hours; no less than 28 hours; no less than 30 hours; no less than 34 hours; no less than 36 hours; no less than 40 hours; no less than 44 hours; or no less than 46 hours.

### 4. Temperature

In various instances, during light exposure, the mixture may be maintained at a temperature of 22° C. to 42° C. In some instances, while being exposed to light, the mixture may be maintained at a temperature of 23° C. to 41° C.; 24° C. to 40° C.; 25° C. to 39° C.; 26° C. to 38° C.; 26° C. to 38° C.; 27° C. to 37° C.; 28° C. to 36° C.; 29° C. to 35° C.; 30° C. to 34° C.; or 31° C. to 32° C. In some instances, during light exposure, the mixture may be maintained at a temperature of no greater than 42° C.; no greater than 40° C.; no greater than 38° C.; no greater than 36° C.; no greater than 34° C.; no greater than 32° C.; no greater than 30° C.; no greater than 28° C.; no greater than 26° C.; or no greater than 24° C. In some instances, during light exposure, the mixture may be maintained at a temperature of no less than 22° C.; no less than 24° C.; no less than 26° C.; no less than 28° C.; no less than 30° C.; no less than 32° C.; no less than 34° C.; no less than 36° C.; no less than 38° C.; or no less than 40° C.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### IV. Experimental Examples

Without limiting the scope of the instant disclosure, various experimental examples of embodiments discussed above were prepared and the results are discussed below.

Abbreviations that may be used in the examples that follow are:
NaOAc is sodium acetate;
KOAc is postassium acetate;
LiOAc is lithium acetate;
Me is methyl;
Et is ethyl;
Ph is phenyl;
Ac is acetyl;
Bn is benzyl;
MeCN is acetonitrile;
THE is tetrahydrofuran;
D₂O is deuterated water;
DMSO is dimethylsulfoxide;
DCE is 1,2-dichloroethane;
DCM is dichloromethane;
DCC is N,N′-dicyclohexylcarbodiimide;
DMF is N,N-dimethylformamide,
DMAP is 4-dimethylaminopyridine;
CDOCO₂D is deuterated glyoxylic acid;
Boc is tert-butyloxycarbonyl;
Ms is methanesulfonyl chloride;
Molar is M;
rt, RT, or r.t. is room temperature;
sat. is saturated;
eq, eq., or equiv is equivalent(s);
wt % or wt. % is weight %;
CFL is compact fluorescent light;
PC is photocatalyst;
4CzIPN is 1,2,3,5-tetrakis(carbazol-9-yl)-4,6-dicyanobenzene;
LED is light emitting diode;
DC is direct current;
TLC is thin layer chromatography;
UV is ultraviolet;
HRMS is high resolution mass spectrometry;
LCMS is liquid chromatography mass spectrometry; and
ESI-TOF is electrospray ionization time-of-flight.

### 1. General Information

Commercially available reagents were purchased from Sigma Aldrich, Matrix Chemical, AKSci, Alfa Aesar, TCI, and Chem Cruz, and used as received unless otherwise noted. Except Isatin, 6-Chloroisatin and 5-Bromoisatin are purchased form Sigma Aldrich, Photosensitizer 4CzIPN is prepared according to corresponding literatures. Merck 60 silica gel was used for chromatography, and Whatman silica gel plates with a fluorescence F254 indicator were used for thin-layer chromatography (TLC) analysis. ¹H and ¹³C NMR spectra were recorded on Bruker Advance 400. Chemical shifts in ¹H NMR spectra are reported in parts per million (ppm) relative to residual chloroform (7.26 ppm) or dimethyl sulfoxide (2.50 ppm) as internal standards. Both 42 Watt Fluorescent CFL Spiral Light Bulbs (TCP 42 Watt Medium Base 277V 2700K) and Purple LED Rope Light (RLL120H-45P, Brilliant Brand Lighting, 395-430 nm) were purchased from Amazon. ¹H NMR data are reported as follows: chemical shift, multiplicity (s=singlet, d=doublet, m=multiplet), coupling constant in Hertz (Hz) and hydrogen numbers based on integration intensities. ¹³C NMR chemical shifts are reported in ppm relative to the central peak of CDCl₃ (77.16 ppm) as internal standards.

### 2. General Procedures and Optimization of Reaction Conditions

### 2.1 General Procedures of Synthesis of 3-formylindoles

To a 20 mL-disposable scintillation vial equipped with a stir bar, were added indole 1 (0.2 mmol, 1 eq), glyoxylic acid 2 (0.6 mmol, 3 eq), and base (0.4 mmol, 2 eq). Then, acetonitrile (2.5 mL) was added using a syringe. The solution was then stirred at room temperature under the irradiation of two 42 W OP-R4-42w CFLs for 48 h using electronic fan to cool the tube. The vial was placed 5 cm away from the light source. After completion of the reaction, 5 mL of water was added and extracted by ethyl acetate (3×15 mL). The combined organic layer was washed with brine (5 mL) and then dried over anhydrous Na₂SO₄ and evaporated in vacuum. The desired products were obtained in the corresponding yields after purification by column chromatography on silica gel eluting with hexane/ethyl acetate or hexane/dichloromethane.

### 2.2 Reaction Conditions Optimization

The investigation of the 3-formylation reaction of indoles commenced with a non-deuterated version glyoxylic acid as formylation agent. After optimization of the reaction conditions (Tables 1 and 2), it was observed that the reaction conducted with 5-methyl indole (1a), glyoxylic acid monohydrate (2, 3.0 equiv) and NaOAc (2.0 equiv) as a base in CH₃CN irradiated by 2×42 W CFL in an open flask at rt for 24 h delivered the desired formylation product in 66% yield (Table 1, entry 1).

TABLE 1
Exploration and optimization^{a}

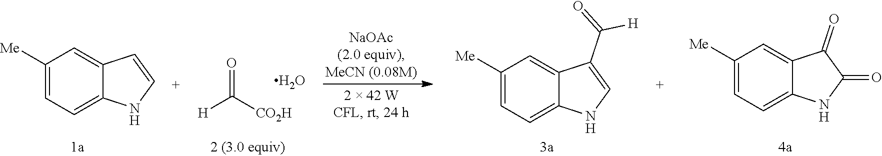

| Entry | Derivation from standard conditions | Yield^{b} (%) |
|---|---|---|
| 1 | none | 66 |
| 2 | 26 W CFL as light source, 0.08M 1a | 32 |
| 3 | 26 W CFL as light source, 0.05M 1a, and 6-Cl-isatin (0.1 equiv) | 32 |
| 4 | DMF as solvent | 27 |
| 5 | 0.05M 1a | 62 |
| 6 | 200 W White LED as light source, 0.08M 1a | 57 |
| 7 | 2 × 30 W Purple LED as light source, 0.08M 1a | 77 |
| 8 | 2 × 40 W Blue LED as light source, 0.08M 1a | 57 |
| 9 | 2 × 7 W 365 nm LED as light source, 0.08M 1a | 65 |
| 10 | 2000 W White LED as light source, 0.08M 1a | 64 |
| 11 | 2 × 14 W CFL as light source, 0.08M 1a | 43 |
| 12 | no O₂ | trace |
| 13 | no light | 0 |
| 14 | no base | 25 |

^{a}Reaction conditions: Unless specified, a mixture of 1a (0.4 mmol), 2a (1.2 mmol), NaOAc (0.8 mmol), in MeCN (0.08M 1a) using 2 × 42 W CFL at rt for
24 h.
^{b}Yield of isolated products.

TABLE 2
Scan of reaction conditions

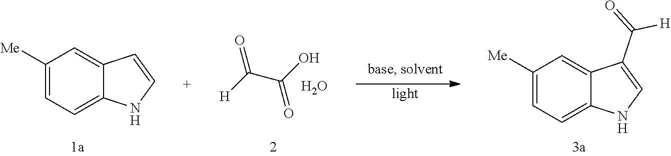

| Entry | Base | Solvent | Time | Yield^{a} | Notes |
|---|---|---|---|---|---|
| 1 | 2 eq NaOAc | THF (0.08M) | 24 h | 0%^{b} | 2 × 42 W CFL, one vial |
| 2 | 2 eq NaOAc | DCM (0.08M) | 24 h | 30% | 2 × 42 W CFL, one vial |
| 3 | 2 eq NaOAc | DCE (0.08M) | 24 h | 30% | 2 × 42 W CFL, one vial |
| 4 | 2 eq NaOAc | DMF (0.08M) | 24 h | 27% | 2 × 42 W CFL, one vial |
| 5 | 2 eq NaOAc | 1,4-dioxane (0.08M) | 24 h | 15% | 2 × 42 W CFL, one vial |
| 6 | 2 eq NaOAc | MeCN (0.08M) | 12 h | 41% | 2 × 42 W CFL, one vial |
| 7 | 2 eq LiOAc | MeCN (0.08M) | 24 h | 32% | 2 × 42 W CFL, one vial |
| 8 | 2 eq NaCO₃ | MeCN (0.08M) | 24 h | 13% | 2 × 42 W CFL, one vial |
| 9 | 2 eq NaHCO₃ | MeCN (0.08M) | 24 h | 17% | 2 × 42 W CFL, one vial |
| 10 | 1 eq NaOAc | MeCN (0.08M) | 24 h | 25% | 2 × 42 W CFL, one vial |
| 11 | 3 eq NaOAc | MeCN (0.08M) | 24 h | 30% | 2 × 42 W CFL, one vial |
| 12 | 2 eq NaOAc | MeCN (0.08M) | 24 h | 66% | 2 × 42 W CFL, one vial |
| 13 | 2 eq NaOAc | MeCN (0.08M) | 24 h | 57% | 200 W White LED, one vial |
| 14 | 2 eq NaOAc | MeCN (0.08M) | 24 h | 77% | 2 × 30 W Purple LED, one vial |
| 15 | 2 eq NaOAc | MeCN (0.08M) | 24 h | 57% | 2 × 40 W Blue LED, one vial |
| 16 | 2 eq NaOAc | MeCN (0.08M) | 24 h | 65% | 2 × 7 W 365 nm LED, one vial |

^{a}Yield is determined by ¹H NMR.
^{b}The yield is calculated after isolation.

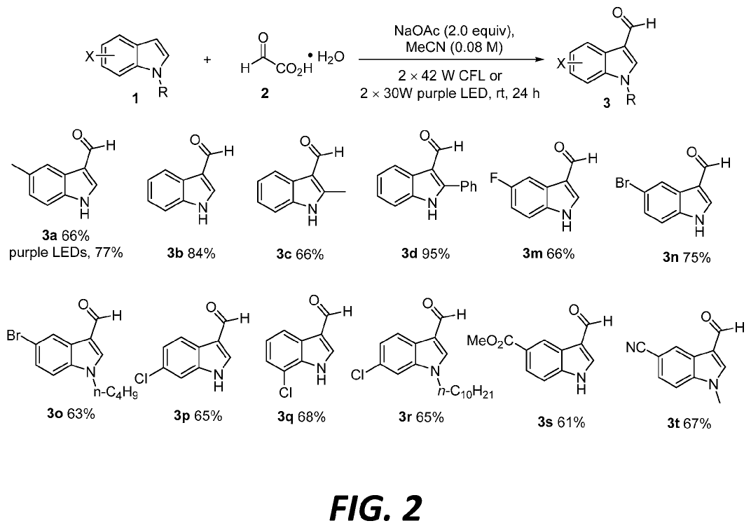

After optimization of the reaction conditions (Tables 1-2), it was discovered that the reaction conducted with 5-methyl indole (1a), glyoxylic acid monohydrate (2, 3.0 equiv) and NaOAc (2.0 equiv) as a base in CH₃CN irradiated by 2×42 W CFL in an open flask at rt for 24 h delivered the desired formylation product in 66% yield (Table 1, entry 1). Furthermore, it was discovered that the protocol was amenable to indoles bearing various substituents (FIG. 2).

### 2.3 General Procedures of Synthesis of Sodium 2-hydroxy-2-(1-methyl-1H-indol-3-yl)acetate

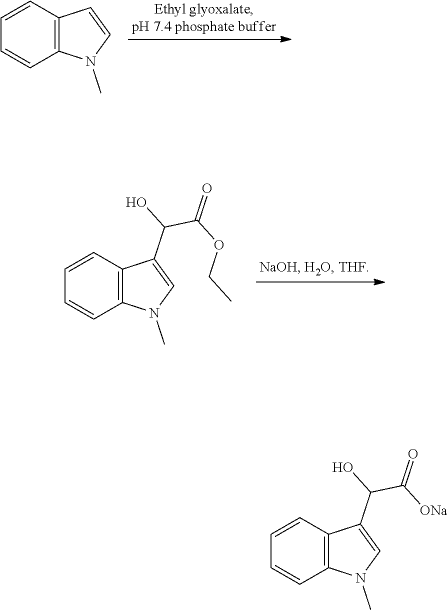

A mixture of indole (0.35 g, 3.0 mmol, 1.0 equiv), ethyl glyoxalate (47% in toluene, 3.0 mL) and pH 7.4 sodium phosphate buffer (0.20 M, 3.0 mL) was vigorously stirred at 25° C. for 4 h. The aqueous layer was extracted with 20 mL of dichloromethane 3 times. The organic layers were combined, dried with Na₂SO₄, and evaporated. The residue was purified by flash column chromatography eluting with gradient of dichloromethane/diethylether (1/0 to 3/2), and afforded 0.48 g of ethyl 2-hydroxy-2-(5-methoxy-1H-indol-3-yl)acetate as white solid (73%).

An aqueous solution of NaOH (150 mg in 0.25 mL H₂O, 3.75 mmol, ca. 1.5 equiv) was added to a solution of 2-(1-(3-azidopropyl)-5-methoxy-1H-indol-3-yl)-2-hydroxyacetate (0.85 g) in THF (7.5 mL). The combined mixture was stirred at 25° C. for 12 h. NaHCO₃ (157 mg, 1.87 mmol, ca. 0.75 equiv) was added to the mixture. The organic solvent of the mixture was evaporated, and the aqueous residue was subjected to flash reverse-phase column chromatography eluting with water. The obtained solution of sodium 2-(1-(3-azidopropyl)-5-methoxy-1H-indol-3-yl)-2-hydroxyacetate (2) was freeze-dried and afforded 0.48 g of sodium 2-hydroxy-2-(1-methyl-1H-indol-3-yl)acetate as white solid (21%, 2 steps).

### 2.4 General Procedures of Synthesis of Deuterated 3-Formylindoles

To a 20 mL-disposable scintillation vial equipped with a stir bar, were added indole 1 (0.2 mmol, 1 eq), deuterated glyoxylic acid 2 (0.6 mmol, 3 eq), and base (0.4 mmol, 2 eq). Then, acetonitrile (2.5 mL) was added using a syringe. The solution was then stirred at room temperature under the irradiation of two 42 W OP-R4-42w CFLs for 48 h using electronic fan to cool the tube. The vial was placed 5 cm away from the light source. After completion of the reaction, 5 mL of water was added and extracted by ethyl acetate (3×15 mL). The combined organic layer was washed with brine (5 mL) and then dried over anhydrous Na₂SO₄ and evaporated in vacuum. The desired products were obtained in the corresponding yields after purification by column chromatography on silica gel eluting with hexane/ethyl acetate or hexane/dichloromethane.

### 2.5 Procedure of Synthesis of 3d-d at a 1.5 mmol Scale

To a 20 mL-disposable scintillation vial equipped with a stir bar, were added indole 1 m (1.5 mmol, 1 eq, 0.29 g), deuterated glyoxylic acid 2 (4.5 mmol, 3 eq, 0.43 g), and NaOAc (0.4 mmol, 2 eq, 0.25 g). Then, acetonitrile (19 mL) was added using a syringe. The solution was then stirred at room temperature under the irradiation of two 42 W OP-R4-42w CFLs for 48 h using electronic fan to cool the tube. The vial was placed 5 cm away from the light source. After completion of the reaction, 20 mL of water was added and extracted by ethyl acetate (3×25 mL). The combined organic layer was washed with brine (20 mL) and then dried over anhydrous Na₂SO₄ and evaporated in vacuum. The desired products were obtained in 82% yield (99% D) after purification by column chromatography on silica gel eluting with hexane/ethyl acetate.

### 3. Substrates Synthesis and Synthetic Application

### 3.1 General Procedure for Synthesis of 1ao-1aq

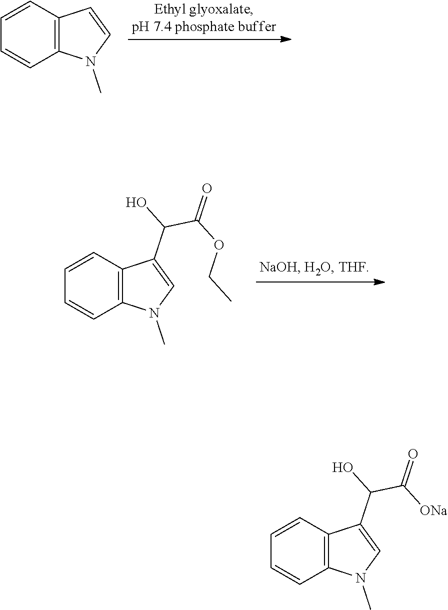

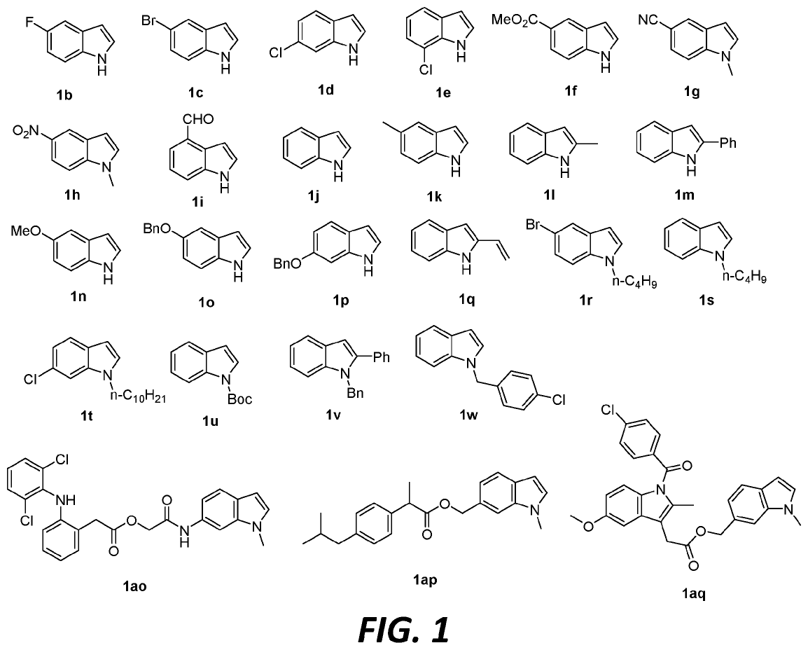

To an oven-dried round-bottom flask with a magnetic stir bar was added acid (0.8 mmol, 1.0 equiv.), (1-methyl-1H-indol-6-yl)methanol (0.142 g, 0.88 mmol, 1.1 equiv.), DCC (0.198 g, 0.96 mmol, 1.2 equiv.) and DMAP (0.098 g, 0.80 mmol, 0.1 equiv.). Dry dichloromethane (4 mL) was added, and the mixture was allowed to stir at room temperature until the acid was consumed (followed by TLC). Typical reaction times were between 0.5 h and 12 h. The white precipitates were filtered off and the solvent was removed under reduced pressure. The desired products were obtained in the corresponding yields after purification by flash chromatography on silica gel eluting with hexane/ethyl acetate or hexane/dichloromethane. Compounds 1ao-1aq are shown in FIG. 1.

For compounds 1a-1an, all these indole derivatives were known compounds. New indole derivatives 1ao, 1ap and 1aq were shown below.

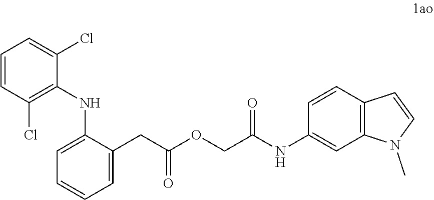 [SMILES: Cn1ccc2ccc(NC(=O)COC(=O)Cc3ccccc3Nc3c(Cl)cccc3Cl)cc21]

The title product was prepared according to the general procedure and purified by column chromatography on silica gel eluting with hexane/ethyl acetate (5:1) to isolate as white solid about 0.33 g (85%).¹H NMR (500 MHz, CDCl₃) δ 7.55 (d, J=8.2 Hz, 1H), 7.32-7.25 (m, 3H), 7.21 (d, J=1.6 Hz, 1H), 7.1-7.0 (m, 3H), 6.91-6.85 (m, 2H), 6.72 (s, 1H), 6.53 (dd, J=8.1, 1.2 Hz, 1H), 6.42 (dd, J=3.1, 0.9 Hz, 1H), 5.27 (s, 2H), 4.66 (s, 2H), 3.90 (s, 2H), 3.62 (s, 3H). ¹³C NMR (126 MHz, CDCl₃) δ 171.6, 167.6, 143.0, 138.0, 136.6, 131.2, 130.0, 129.7, 129.0, 128.9, 128.3, 128.2, 124.3, 124.2, 122.3, 121.2, 120.4, 118.6, 110.1, 101.1, 68.6, 61.5, 60.5, 38.2, 32.9, 21.2, 14.4.

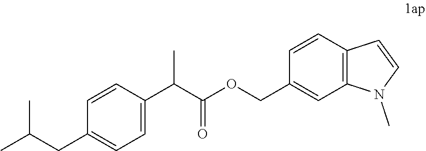 [SMILES: CC(C)Cc1ccc(C(C)C(=O)OCc2ccc3ccn(C)c3c2)cc1]

The title product was prepared according to the general procedure and purified by column chromatography on silica gel eluting with hexane/ethyl acetate (8:1) to isolate as white solid about 0.24 g (87%). ¹H NMR (500 MHz, CDCl₃) δ 7.60 (d, J=8.1 Hz, 1H), 7.34-7.28 (m, 2H), 7.2 (s, 1H), 7.17-7.13 (m, 2H), 7.11-7.03 (m, 2H), 6.52-6.43 (m, 1H), 5.33 (q, J=12.1 Hz, 2H), 3.84 (q, J=7.1 Hz, 1H), 3.76 (s, 3H), 2.52 (d, J=7.2 Hz, 2H), 2.11 (s, 1H), 1.93 (dq, J=13.6, 6.8 Hz, 2H), 1.53 (d, J=7.2 Hz, 3H), 0.95 (d, J=6.8 Hz, 7H). ¹³C NMR (126 MHz, CDCl₃) δ 174.7, 140.5, 138.0, 136.6, 129.6, 129.5, 129.4, 128.5, 127.4, 120.9, 119.9, 109.3, 101.0, 67.5, 45.3, 45.2, 32.8, 30.3, 30.3, 22.5, 18.7.

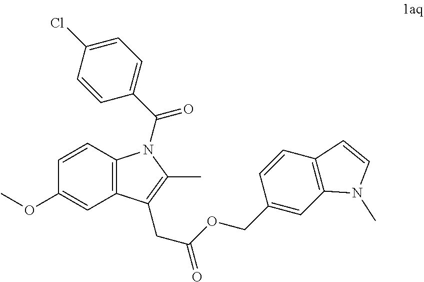

The title product was prepared according to the general procedure and purified by column chromatography on silica gel eluting with hexane/ethyl acetate (3:1) to isolate as white solid about 0.34 g (86%). ¹H NMR (500 MHz, CDCl₃) δ 7.61-7.55 (m, 3H), 7.41-7.39 (m, 2H), 7.22 (s, 1H), 7.05-7.03 (m, 2H), 6.92-6.89 (m, 2H), 6.65 (dd, J=9.0, 2.5 Hz, 1H), 6.45 (dd, J=3.1, 0.9 Hz, 1H), 5.26 (s, 2H), 3.70-3.68 (m, 7H), 2.32 (s, 3H), 1.53 (s, 14H). ¹³C NMR (126 MHz, CDCl₃) δ 156.1, 131.1, 129.7, 129.1, 129.0, 120.9, 120.0, 115.0, 111.9, 109.6, 101.2, 101.0, 67.9, 55.6, 32.8, 30.6, 13.5.

### 3.2 General Procedure for Synthesis of Deuterated Glyoxylic Acid (2-d)

To anhydrous oxalic acid (4.5 g, 50 mmol) in a 25 mL flask, was added 5 mL of heavy water (D₂O). The mixture was heated to 90° C., and stirred until the oxalic acid was completely dissolved, then cooled to room temperature. The flask was sealed, and kept in the refrigerator at 0-5° C. for 3 h. The cooling was vacuumed quickly a diaphragm pump and, slowly warmed up to room temperature, and then was heated slowly to 90° C. with an oil bath. The heating speed must be slow to avoid bumping. After the temperature rose to 90° C., it was kept it 2 h until it became a white color powdered solid. The above procedure was repeated to give deuterated oxalic acid with a deuteration level at 97.2%.

Lead sheet with a thickness of 0.5˜1 mm was cut into strips about 0.5 cm wide and 5 cm long, and then polished with fine sandpaper and ordinary white paper. The pretreated lead was used as electrolytic electrodes. The deuterated oxalic acid (about 4.5 g, 50 mmol) was dissolved 8 mL of heavy water in an electrolytic vial, heated to completely dissolve, and then slowly cooled to room temperature with crystals formed and kept for 1-2 h until the crystals were no longer formed. The resulting mixture was used as an electrolytic mixture and was placed in a vial. The above freshly prepared lead sheets were used as electrodes. A DC stabilized power supply was used to control electrolysis voltage. Thus, an electrolysis device was set up. The distance between the two lead electrodes in the vial is about 1 cm. The large distance reduces the electrolysis efficiency, as short distances may induce short circuiting, thereby reducing the electrolysis yield. A small magnetic stirrer was placed in the electrolysis vial and the stirring speed was moderated during the electrolysis. The stirring speed was set at a moderate speed. The electrolysis voltage was set at 4˜5 V. The electrolysis time is 6-10 h, based on the complete disappearance of the oxalic acid solid in the electrolysis bottle. After the oxalic acid was consumed, the electrolysis continued for an additional 1 h. At this time, the current was significantly reduced, and the electrolysis was stopped to obtain a mixture of deuterated glyoxylic acid (the electrolyzed electrode must be pure otherwise current will obviously decrease when oxalic acid is still present during the electrolysis process). After electrolysis, the electrolytic mixture was frozen in a refrigerator at −20° C. for 3 h, and then dried with a diaphragm pump or an oil pump until the weight was no longer reduced, and a moist solid deuterated glyoxylic acid (2-d) was obtained. The deuterated glyoxylic acid (2-d) was directly used for the formylation reaction. The moist solid was then freeze-dried 3-4 times. The deuteration rate (95-99%) of the synthesized deuterated glyoxylic acid was determined by converting the product to ethyl 2-oxoacetate-d (see section 3.3). 3.3 Synthesis of Ethyl 2-Oxoacetate-d to Confirm Deuteration of Glyoxylic Acid

To a stirred solution of ethanol (0.1 mmol, 1.0 equiv) and deuterated glyoxylic acid (2-d, 2.0 equiv) and pyridine (5.0 equiv) in dry THF was added MsCl (2.4 equiv) dropwise at 0° C. and stirred for 2 h at room temperature. Then the mixture was quenched by 10% citric acid aqueous solution, extracted with Et₂O. The combined organic layers were washed with brine and dried by anhydrous Na₂SO₄. The organic phase was concentrated under reduced pressure and the residue was purified by flash column chromatography to get ethyl 2-oxoacetate-d. This study is schematically illustrated below.

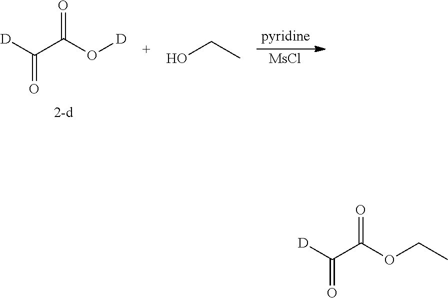

TABLE 3
Exploration and Optimization

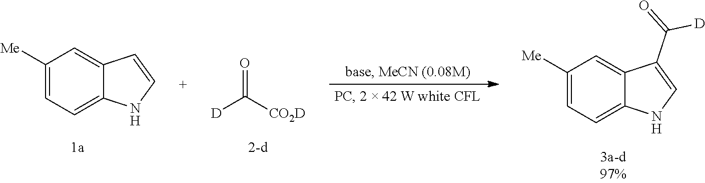

| Entry | Base (equiv) | Photocatalyst | Yield^{b} (%) |
|---|---|---|---|
| 1 | NaOAc (2.0) | none | 31% |
| 2 | LiOAc (2.0) | none | 23% |
| 3 | NaOAc (2.0) | Rose bengal (2 mol %) | 26% |
| 4 | NaOAc (2.0) | Eosin Y (2 mol %) | 24% |
| 5 | NaOAc (2.0) | Isatin (10% mol) | 65% |
| 6 | NaOAc (2.0) | Isatin (10 mol %) | 59% |
| 7 | NaOAc (2.0) | 5-Cl-Isatin (10% mol) | 61% |
| 8 | NaOAc (2.0) | 6-Cl-Isatin (10% mol) | 71% |
| 9 | NaOAc (2.0) | 6-Cl-Isatin (10% mol) | 72%^{c} |
| 10^{d} | NaOAc (2.0) | none | 70% |

^{a}Reaction conditions: Unless specified, a mixture of 1a (0.4 mmol), 2-d (1.2 mmol), NaOAc (0.8 mmol) in MeCN (0.08M 1a)
using 2 × 42 W CFL at rt for 24 h.
^{b}Isolated yield.
^{c}2 × 30 W purple LED applied.
^{d}Freeze dried CDOCO₂D used.

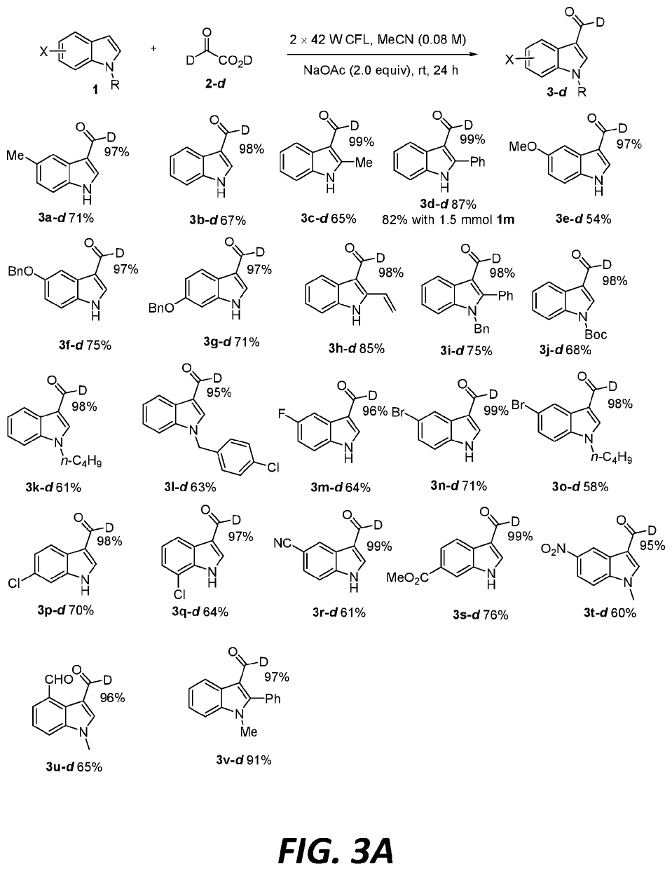

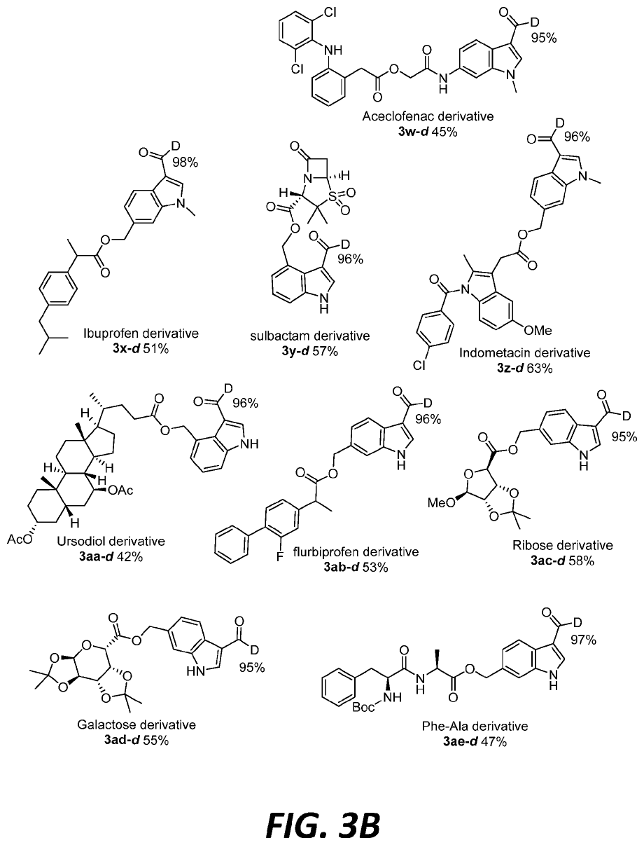

As shown in FIG. 3A, the optimized reaction conditions serve as a general approach to the synthesis of a wide array of C1-deuterated 3-formylindoles. In all cases studied, excellent level of deuteration (95-99%) is obtained. Furthermore, the reaction yield is not affected by electronic effect. The substrates bearing electron-neutral (3b-d), -donating (3a-d, 3e-d, 3f-d), and -withdrawing (3m-d-3w-d) substituents on the indole ring amenable to the process. The steric effect is also limited when 2-methyl, 2-pheny groups are installed in the adjacent formylation position (3c-d, 3d-d). Furthermore, various ‘N’ protecting forms including free NH (3a-d-3h-d), N-Boc (3j-d) and N-alkyl (3k-d, 3o-d) in the indole structure can be tolerated. Finally, late-stage formylation of more challenging targets of complex biologically active molecules including clinically used therapeutics aceclofenac, ibuprofen, sulbactam, flurbiprofen and indometacin derivatives ursodiol, saccharides and dipeptides can be achieved to give the desired products 3y-d-3af-d in good yields (42-63%) with excellent deuterium incorporation (FIG. 3B). The protocol can be scaled up at 1.5 mmol without significant loss reaction yield (82% yield) and deuteration level (99%), as shown for the synthesis of 3d-d above.

The new photochemical mediated formylation reaction involves an unprecedented aldol-oxidative decarboxylation cascade process. In previous indole formylation reactions, an additional amine was used to activate the aldehyde group of glyoxylic acid for a Mannich reaction with indoles to in situ generate an amine adduct for a subsequent oxidative formylation. These studies have uncovered that a direct aldol reaction of glyoxylic acid with indoles is possible without requiring an additional amine as catalyst.

### 5. Mechanistic Studies

### 5.1 Measurement of Cyclic Voltammogram

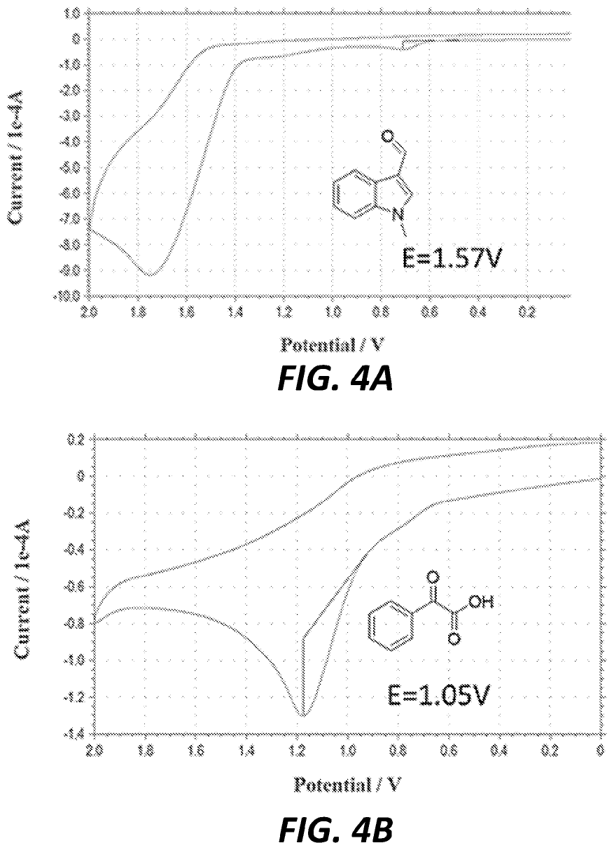

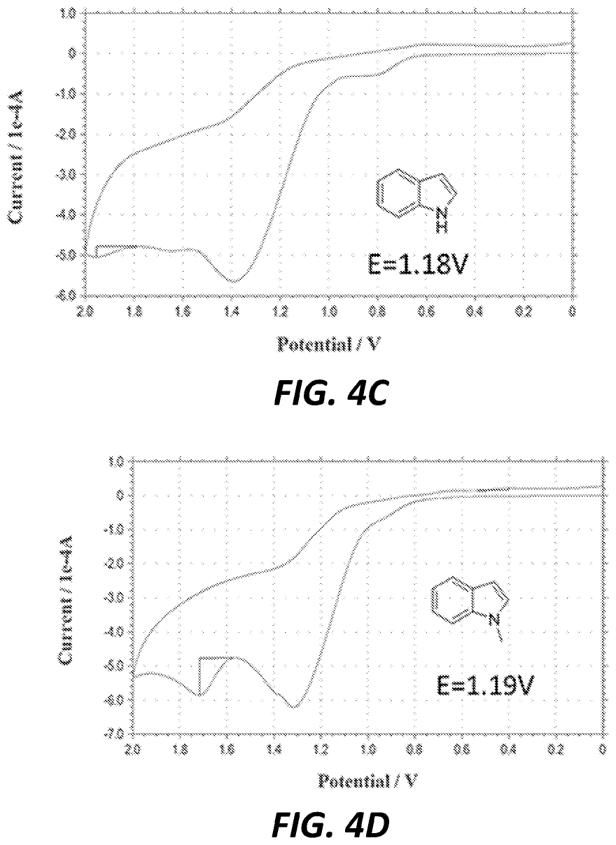

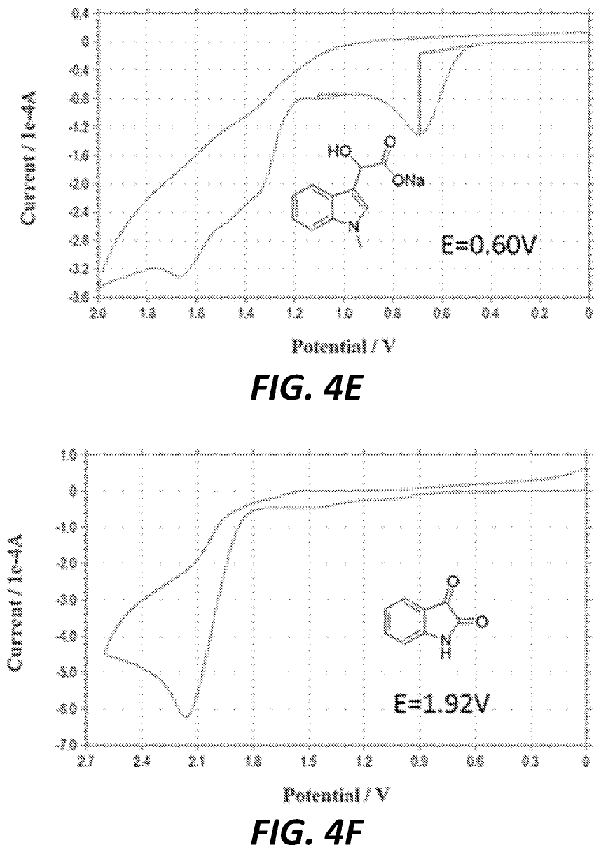

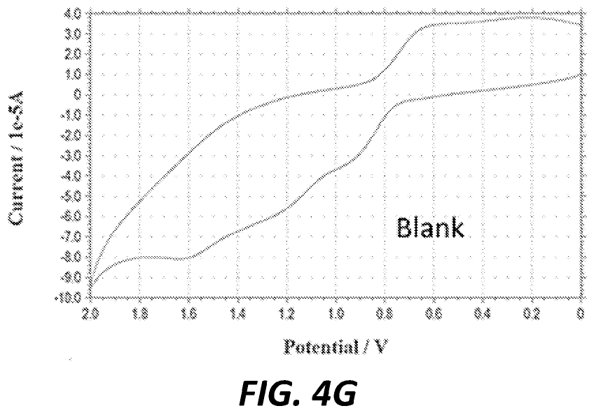

Voltammetric measurements were recorded on a CH Instruments: Model 600E Series Electrochemical Analyzer using a standard three electrodes setup in dry and degassed MeCN (10 mL), with ferrocene as an internal reference (E_{1/2}^{ox}=+0.40 V vs SCE) and Bu₄NPF₆ as the electrolyte (0.10 mmol). Cyclic voltammograms were recorded at a scan rate of 0.2 V/s (FIGS. 4A-4G).

### 5.2 Control Study of the Dark and N₂ Atmosphere Condition

A light activated electron-donor-acceptor (EDA) originated from N-methyl-indole (donor) and glyoxylic acid 2 (acceptor) was initially proposed.

As shown in Scheme 1 below, the analysis was carried out with 0.1 mmol of 4a and 0.3 mmol of glyoxylic acid monohydrate in 2.5 mL of CDCl₃. The solution was then stirred at room temperature. The analysis was carried out with 0.1 mmol of 4a and 0.3 mmol of glyoxylic acid monohydrate in 2.5 mL of CDCl₃. The mixture was degassed by freeze-pump-thaw method, then sealed with parafilm. The solution was then stirred at room temperature under the irradiation of two 42 W OP-R4-42w CFLs using electronic fan to cool the tube.

### Scheme 1

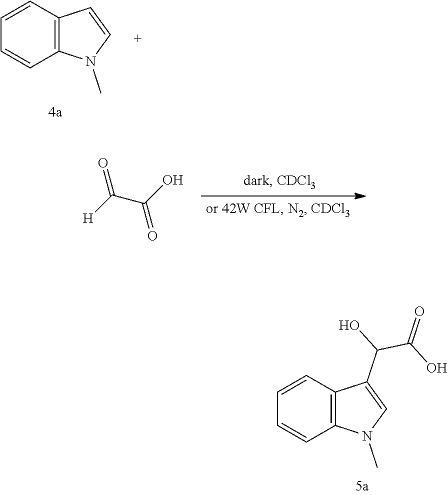

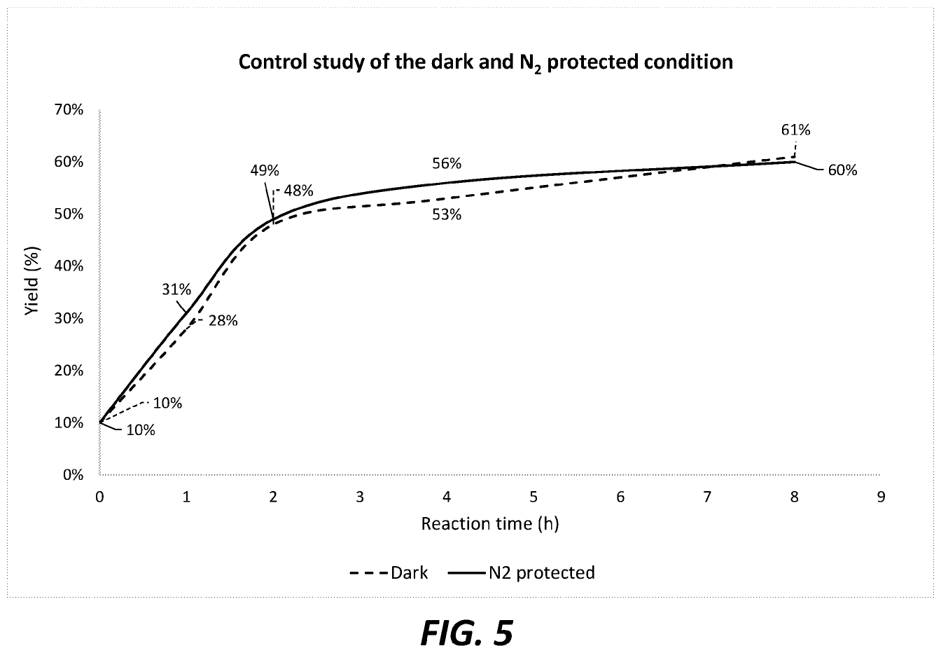

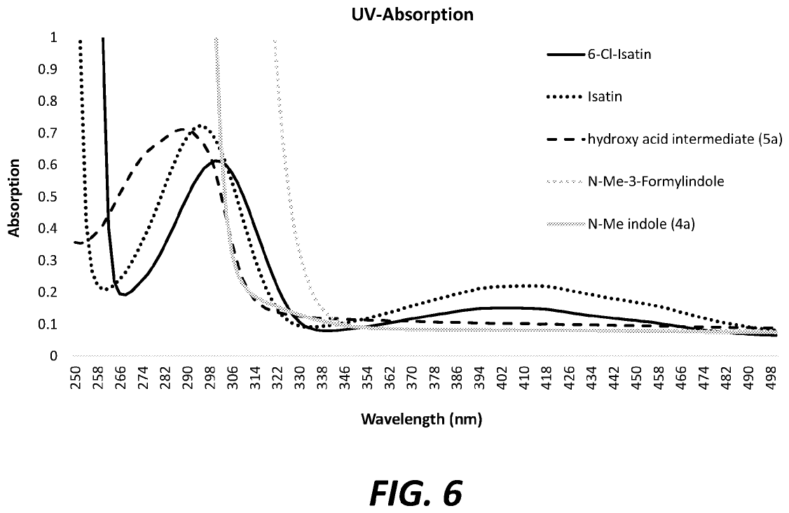

The results for the dark and N₂ protected conditions are shown in FIG. 5. The photophysical properties of the indoles, isatins were also recorded in MeCN (FIG. 6).

### 5.4 General Procedure for Synthesis of 3× and 5×

To a 20 mL-disposable scintillation vial equipped with a stir bar, were added 5-Bromo-1-methylindole (0.2 mmol, 1 eq), glyoxylic acid (0.4 mmol, 2 eq), and NaOAc (0.4 mmol, 2 eq). Then, acetonitrile (2.5 mL) was added using a syringe. The solution was then stirred at room temperature under the irradiation of two 42 W OP-R4-42w CFLs for 48 h using electronic fan to cool the tube. The vial was placed 5 cm away from the light source. After completion of the reaction, 5 mL of water was added and extracted by ethyl acetate (3×15 mL). The combined organic layer was washed with brine (5 mL) and then dried over anhydrous Na₂SO₄ and evaporated in vacuum. The desired products were obtained in the corresponding yields after purification by column chromatography on silica gel eluting with hexane/ethyl acetate or hexane/dichloromethane.

### 5.5 Elucidating Reaction Mechanism Using 3× and 5×

Scheme 2 shows the designed experiments for elucidating the reaction mechanism.

### Scheme 2

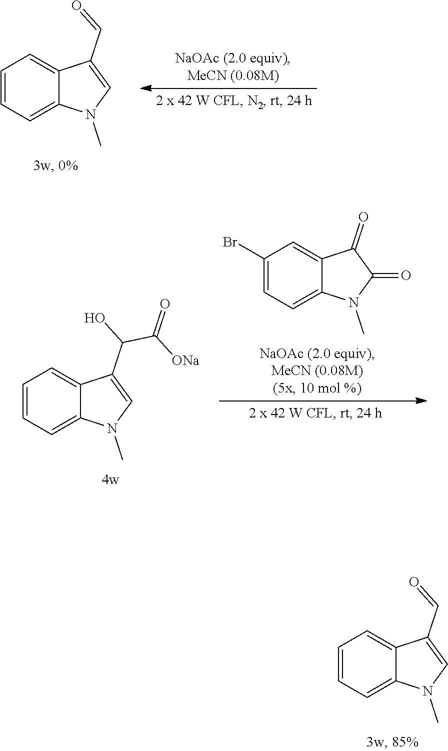

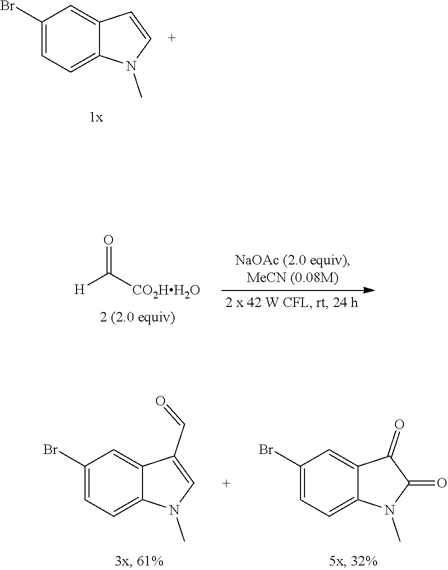

In the studies shown in Scheme 2 above, the formylation reaction was carried out without an external PC. It was observed that the isatins were formed without need of an external PC and their formation was indole structure dependent. The corresponding isatin product was isolated with indoles bearing electron withdrawing groups such as 5-Br, 5-CO₂Me and 6-CN on the benzene ring. For example, 5-Br-N-methyl-isatin 5× was obtained in 32% yield in addition to 61% formylation product 3× under the standard reaction conditions (Scheme 3a). It was hypothesized that the isatin byproduct might function as the PC for the oxidative decarboxylation-formylation process. N-methyl isatin with strong oxidation power (E^{ox}=+1.91 V vs SCE) is excited by light and enables the oxidative decarboxylation of α-hydroxy carboxylate (E^{red}=+0.60 V vs SCE) (FIG. 4E). Indeed, in a control experiment, irradiation of the preformed aldol adduct 4w by 2×42W CFL did not produce formylation product 3w in the absence of 5x. In contrast, in the presence of 5-bromo-isatin (10 mol %), 4w was efficiently transformed into the formylation product 3w in 85% yield (Scheme 2). This confirms the formylation reaction undergoes an unprecedented aldol-oxidative decarboxylation cascade process.

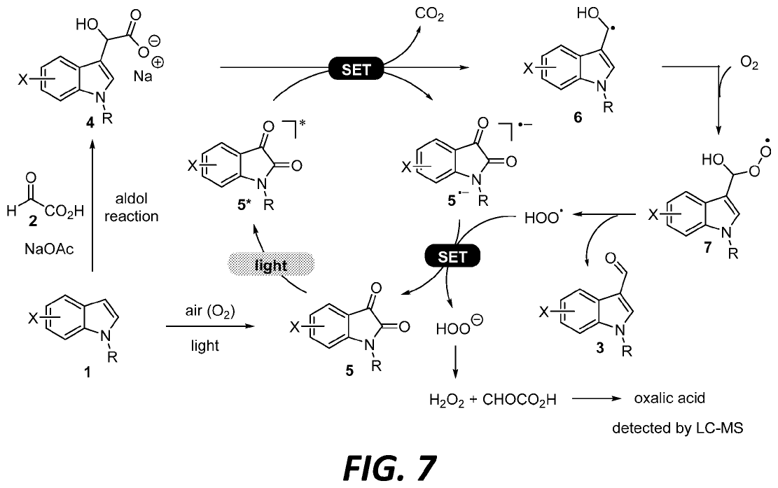

The studies illustrated in Scheme 2 in conjunction with the identification of isatin byproduct as a PC suggested a plausible mechanism for the new formylation process as illustrated by FIG. 7. Aldol reaction of indole 1 with glyoxylic acid 2 generates α-hydroxy acid sodium salt intermediate 4. Meanwhile, a small percentage of the indole is oxidized by air (O₂) to byproduct isatin 5 under visible light irritation. The excited isatin 5* serves as PC for the oxidative decarboxylation of 4 to give radical anion 5′ and radical 6. The resulting radical 6 then reacts with O₂ to form a peroxide radical 7. Fragmentation of the radical 7 gives rise to formylation product 3 and hydrogen peroxide anion. The formation of H₂O₂ mediated oxidation byproduct oxalic acid from glyoxylic acid, detected by LC-MS.

In summary, a mild metal- and oxidant-free visible-light photoredox mediated selective C3-formylation of indoles has been developed. The new uncovered process is synthetically sustainable by using readily accessible indoles and feedstock glyoxylic acid as the formylation reagent, molecular oxygen (air) as the terminal oxidant with visible light without requiring external PC and additional amine catalyst. A new self-activation mode by the generation of byproduct isatin as PC is found. The synthetic strategy has been successfully adopted for the practical synthesis of C1-deuterated 3-formyl indoles. A cost-effective deuterated glyoxylic acid as new formyl deuteration reagent has been developed for this demand. The mild, operationally simple protocol serves as a general preparing power for the practical synthesis of structurally diverse C1-deuerated 3-formyl indoles with broad functional group tolerance and late-stage deuteration of complex structures at high level (95-99%).

### 6. Compound Characterization Data

### 5-Methyl-1H-indole-3-carbaldehyde (3a)

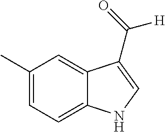

The title product was prepared according to the general procedure and purified by column chromatography on silica gel eluting with hexane/ethyl acetate (5:1) to isolate as white solid about 17 mg (77%). ¹H NMR (500 MHz, CDCl₃) δ 9.93 (s, 1H), 8.07-7.96 (m, 1H), 7.72 (d, J=3.1 Hz, 1H), 7.25 (d, J=8.3 Hz, 1H), 7.05 (dd, J=8.4, 1.7 Hz, 1H), 2.38 (s, 3H). ¹³C NMR (126 MHz, CDCl₃) δ 185.4, 136.0, 135.1, 132.9, 126.0, 124.7, 121.6, 119.2, 111.3, 21.5. HRMS (ESI-TOF) m/z: [C₁₀H₉NO+H]⁺ calcd for C₁₀H₁₀NO: 160.0757, found: 160.0756.

### 1H-Indole-3-carbaldehyde (3b)

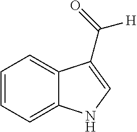 [SMILES: O=Cc1c[nH]c2ccccc12]

The title product was prepared according to the general procedure and purified by column chromatography on silica gel eluting with hexane/ethyl acetate (5:1) to isolate as white solid about 14 mg (84%). ¹H NMR (500 MHz, DMSO) δ 12.13 (s, 1H), 9.95 (s, 1H), 8.29 (s, 1H), 8.11 (d, J=7.68 Hz, 1H), 7.52 (d, J=7.96 Hz, 1H), 7.33-7.12 (m, 2H). ¹³C NMR (126 MHz, DMSO-d₆) δ 185.4, 138.9, 137.5, 124.6, 123.9, 122.6,121.3, 118.6, 112.9. FIRMS (ESI-TOF) m/z: [C₉H₇NO+H]⁺ calcd for C₉H₈NO: 146.0600, found: 146.0600.

### 2-Methyl-1H-indole-3-carbaldehyde (3c)

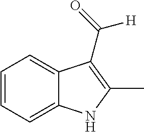

The title product was prepared according to the general procedure and purified by column chromatography on silica gel eluting with hexane/ethyl acetate (5:1) to isolate as white solid about 18 mg (66%). ¹H NMR (500 MHz, DMSO) δ 11.98 (s, 1H), 10.06 (s, 1H), 8.05 (dd, J=6.7, 2.1 Hz, 1H), 7.39 (dd, J=6.8, 2.0 Hz, 1H), 7.17 (tt, J=7.2, 5.6 Hz, 2H), 2.69 (s, 3H). ¹³C NMR (126 MHz, DMSO-d₆) δ 184.7, 149.0, 135.8, 126.0, 123.1, 122.3, 120.4, 114.1, 111.8, 11.9. HRMS (ESI-TOF) m/z: [C₁₀H₉NO+H]⁺ calcd for C₁₀H₁₀NO: 160.0757, found: 160.0755.

### 2-Phenyl-1H-indole-3-carbaldehyde (3d)

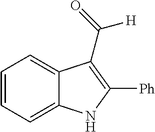 [SMILES: O=Cc1c(-c2ccccc2)[nH]c2ccccc12]

The title product was prepared according to the general procedure and purified by column chromatography on silica gel eluting with hexane/ethyl acetate (5:1) to isolate as colorless oil about 10 mg (95%). ¹H NMR (500 MHz, DMSO) δ 12.40 (s, 1H), 9.98 (s, 1H), 8.22 (d, J=7.75 Hz, 1H), 7.83-7.72 (m, 2H), 7.61 (qd, J=7.61, 3.64 Hz, 3H), 7.52 (d, J=7.91 Hz, 1H), 7.35-7.18 (m, 2H). ¹³C NMR (126 MHz, DMSO-d₆) δ 186.0, 149.6, 136.4, 130.4 (2C), 130.3, 130.3, 129.5 (2C), 126.2, 124.2, 122.9, 121.5, 113.9, 112.5. HRMS (ESI-TOF) m/z: [C₁₅H₁₁NO+H]⁺ calcd for C₁₅H₁₂NO: 222.0913, found: 222.0910.

### 5-Fluoro-1H-indole-3-carbaldehydee (3m)

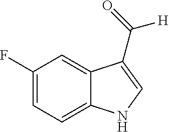 [SMILES: O=Cc1c[nH]c2ccc(F)cc12]

The title product was prepared according to the general procedure and purified by column chromatography on silica gel eluting with hexane/ethyl acetate (5:1) to isolate as white solid about 15 mg (66%). ¹H NMR (500 MHz, DMSO-d₆) δ 9.93 (s, 1H), 8.36 (s, 1H), 7.77 (dd, J=9.6, 2.6 Hz, 1H), 7.53 (dd, J=8.8, 4.5 Hz, 1H), 7.12 (td, J=9.2, 2.7 Hz, 1H). ¹³C NMR (126 MHz, DMSO-d₆) δ 185.5, 159.2 (d, J=235.6 Hz), 140.1, 134.1, 125.2 (d, J=11.3 Hz), 118.6 (d, J=3.8 Hz), 114.2 (d, J=10.1 Hz), 112.1 (d, J=25.2 Hz), 106.1 (d, J=24.0 Hz). ¹⁹F NMR (376 MHz, DMSO) δ-121.19 (td, J=9.5, 4.6 Hz). HRMS (ESI-TOF) m/z: [C₉H₆FNO+H]⁺ calcd for ₉H₇FNO: 164.0506, found: 164.0503.

### 5-Bromo-1H-indole-3-carbaldehyde (3n)

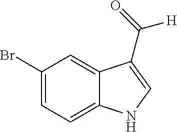 [SMILES: O=Cc1c[nH]c2ccc(Br)cc12]

The title product was prepared according to the general procedure and purified by column chromatography on silica gel eluting with hexane/ethyl acetate (5:1) to isolate as white solid about 14 mg (75%). ¹H NMR (500 MHz, DMSO-d₆) δ 12.30 (s, 1H), 9.93 (s, 1H), 8.35 (s, 1H), 8.23 (d, J=2.0 Hz, 1H), 7.50 (d, J=8.6 Hz, 1H), 7.40 (dd, J=8.6, 2.1 Hz, 1H). ¹³C NMR (126 MHz, DMSO-d₆) δ 185.6, 139.7, 136.2, 126.53, 126.4, 123.4, 117.9, 115.3, 115.0. HRMS (ESI-TOF) m/z: [C₉H₆BrNO+H]⁻ calcd for C₉H₇BrNO: 223.9706, found: 223.9703.

### 5-Bromo-1-butyl-1H-indole-3-carbaldehyde (30)

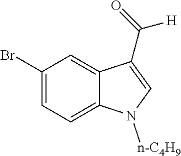

The title product was prepared according to the general procedure and purified by column chromatography on silica gel eluting with hexane/ethyl acetate (5:1) to isolate as white solid about 18 mg (63%). ¹H NMR (500 MHz, CDCl₃) δ 9.94 (s, 1H), 8.45 (d, J=1.91 Hz, 1H), 7.70 (s, 1H), 7.40 (dd, J=8.74, 1.94 Hz, 1H), 7.23 (d, J=8.67 Hz, 1H), 4.15 (t, J=7.18 Hz, 2H), 1.90-1.82 (m, 2H), 1.41-1.32 (m, 2H), 0.96 (t, J=7.37 Hz, 3H). ¹³C NMR (126 MHz, CDCl₃) δ 184.3, 138.8, 135.9, 126.9, 124.8, 117.4, 116.5, 115.8, 111.5, 47.2, 31.7, 20.0, 13.6. HRMS (ESI-TOF) m/z: [C₁₃H₁₄BrNO+H]⁺ calcd for C₁₃H₁₅BrNO: 280.0332, found: 280.0336.

### 6-Chloro-1H-indole-3-carbaldehyde (3p)

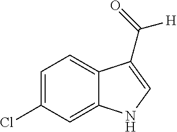 [SMILES: O=Cc1c[nH]c2cc(Cl)ccc12]

The title product was prepared according to the general procedure and purified by column chromatography on silica gel eluting with hexane/ethyl acetate (5:1) to isolate as white solid about 24 mg (65%). ¹H NMR (500 MHz, DMSO-d₆) δ 12.21 (s, 1H), 9.94 (s, 1H), 8.34 (d, J=3.1 Hz, 1H), 8.08 (d, J=8.4 Hz, 1H), 7.58 (d, J=1.9 Hz, 1H), 7.25 (dd, J=8.4, 1.9 Hz, 1H). ¹³C NMR (126 MHz, DMSO-d₆) δ 185.6, 139.7, 138.0, 128.4, 123.3, 122.9, 122.6, 118.4, 112.7. HRMS (ESI-TOF) m/z: [C₉H₆ClNO+H]⁺ calcd for C₉H₇ClNO: 180.0211, found: 180.0213.

### 7-Chloro-1H-indole-3-carbaldehyde (3q)

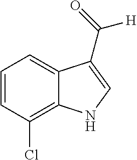 [SMILES: O=Cc1c[nH]c2c(Cl)cccc12]

The title product was prepared according to the general procedure and purified by column chromatography on silica gel eluting with hexane/ethyl acetate (5:1) to isolate as white solid about 25 mg (68%). ¹H NMR (500 MHz, CDCl₃) δ 10.06 (s, 1H), 8.20 (dd, J=7.9, 1.0 Hz, 1H), 7.88 (d, J=2.2 Hz, 1H), 7.31 (dd, J=7.7, 1.0 Hz, 1H), 7.24 (s, 1H). ¹³C NMR (126 MHz, CDCl₃) δ 185.2, 160.3, 139.6, 135.2, 125.8, 123.9, 123.8, 120.6, 116.9. HRMS (ESI-TOF) m/z: [C₉H₆ClNO+H]⁺ calcd for C₉H₇ClNO: 180.0211, found: 180.0215.

### 6-Chloro-1-decyl-1H-indole-3-carbaldehyde (3r)

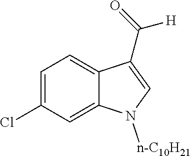

The title product was prepared according to the general procedure and purified by column chromatography on silica gel eluting with hexane/ethyl acetate (5:1) to isolate as white solid about 16 mg (65%). ¹H NMR (500 MHz, CDCl₃) δ 9.84 (s, 1H), 8.10 (d, J=8.42 Hz, 1H), 7.58 (s, 1H), 7.24 (d, J=1.96 Hz, 1H), 7.17-7.13 (m, 1H), 3.99 (t, J=7.27 Hz, 2H), 1.75 (t, J=7.21 Hz, 2H), 1.14 (d, J=7.67 Hz, 15H), 0.77 (d, J=6.72 Hz, 3H). ¹³C NMR (126 MHz, CDCl₃) δ 184.4, 138.8, 137.7, 129.9, 123.9, 123.5, 123.1, 118.0, 110.2, 47.4, 31.8, 29.7, 29.5, 29.4, 29.3, 29.1, 26.8, 22.7, 14.1. HRMS (ESI-TOF) m/z: [C₁₉H₂₆ClNO+H]⁺ calcd for C₁₉H₂₇ClNO: 320.1776, found: 320.1778.

### Methyl 3-formyl-1H-indole-5-carboxylate (3s)

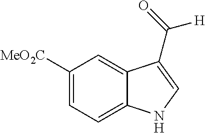 [SMILES: COC(=O)c1ccc2[nH]cc(C=O)c2c1]

The title product was prepared according to the general procedure and purified by column chromatography on silica gel eluting with hexane/ethyl acetate (4:1) to isolate as white solid about 29 mg (61%). ¹H NMR (500 MHz, DMSO-d₆) δ 12.43 (s, 1H), 9.99 (s, 1H), 8.78 (dd, J=1.7, 0.7 Hz, 1H), 8.42 (s, 1H), 7.88 (dd, J=8.5, 1.8 Hz, 1H), 7.61 (dd, J=8.5, 0.8 Hz, 1H), 5.74 (s, 1H), 3.88 (s, 3H). ¹³C NMR (126 MHz, DMSO-d₆) δ 185.8, 140.6, 140.1, 124.9, 124.2, 124.0, 123.5, 119.2, 113.0, 52.4. HRMS (ESI-TOF) m/z: [C₁₁H₉NO₃+H]⁺ calcd for C₁₁H₁₀NO₃: 204.0655, found: 204.0654.

### 3-Formyl-1-methyl-1H-indole-5-carbonitrile (3t)

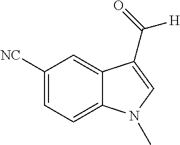

The title product was prepared according to the general procedure and purified by column chromatography on silica gel eluting with hexane/ethyl acetate (5:1) to isolate as white solid about 20 mg (67%). ¹H NMR (400 MHz, CDCl₃) δ 10.03 (s, 1H), 8.74-8.63 (m, 1H), 7.83 (s, 1H), 7.60 (dd, J=8.6, 1.6 Hz, 1H), 7.46 (d, J=8.5 Hz, 1H), 3.96 (s, 3H). ¹³C NMR (101 MHz, CDCl₃) δ 184.1, 140.6, 139.3, 127.5, 127.1, 125.0, 119.7, 118.3, 110.9, 106.4, 34.0. HRMS (ESI-TOF) m/z: [C₁₁HsN₂O+H]⁺ calcd for C₁₁H₉N₂O: 185.0709, found: 185.0713.

### 5-Methyl-1H-indole-3-carbaldehyde-d (3a-d)

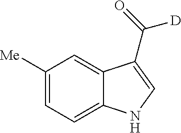 [SMILES: [2H]C(=O)c1c[nH]c2ccc(C)cc12]

The title product was prepared according to the general procedure and purified by column chromatography on silica gel eluting with hexane/ethyl acetate (5:1) to isolate as white soild about 16 mg (71%). ¹H NMR (500 MHz, DMSO) δ 12.02 (s, 1H), 9.90 (s, 0.03H) 8.22 (d, J=3.21 Hz, 1H), 7.91 (d, J=1.78 Hz, 1H), 7.39 (d, J=8.22 Hz, 1H), 7.08 (dd, J=8.33, 1.74 Hz, 1H), 2.41 (s, 3H). ¹³C NMR (126 MHz, DMSO-d₆) δ 185.3-184.9 (m), 138.8, 135.8, 131.5, 125.4, 124.9, 121.1, 118.2, 112.5, 21.7. HRMS (ESI-TOF) m/z: [C₁₀H₈DNO+H]⁺ calcd for C₁₀H₉DNO: 161.0820, found: 161.0819.

### 1H-Indole-3-carbaldehyde-d (3b-d)

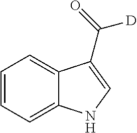 [SMILES: [2H]C(=O)c1c[nH]c2ccccc12]

The title product was prepared according to the general procedure and purified by column chromatography on silica gel eluting with hexane/ethyl acetate (5:1) to isolate as light colorless oil about 20 mg (67%). ¹1H NMR (500 MHz, DMSO) δ 12.12 (s, 1H), 9.94 (s, 0.01H) 8.29 (d, J=3.12 Hz, 1H), 8.10 (d, J=7.68 Hz, 1H), 7.51 (d, J=7.97 Hz, 1H), 7.36-7.15 (m, 2H). ¹³C NMR (126 MHz, DMSO-d₆) δ 185.7-185.2 (m), 138.9, 137.5, 124.6, 123.9, 122.6, 121.3, 118.6, 112.9. HRMS (ESI-TOF) m/z: [C₉H₆DNO+H]⁺ calcd for C₉H₇DNO: 147.0663, found: 147.0664.

### 2-Methyl-1H-indole-3-carbaldehyde-d (3c-d)

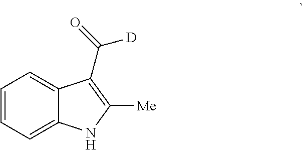 [SMILES: [2H]C(=O)c1c(C)[nH]c2ccccc12]

The title product was prepared according to the general procedure and purified by column chromatography on silica gel eluting with hexane/ethyl acetate (5:1) to isolate as white soild about 19 mg (65%). ¹H NMR (500 MHz, DMSO) δ 11.98 (s, 1H), 10.06 (s, 0.01H) 8.05 (dd, J=6.67, 2.10 Hz, 1H), 7.39 (dd, J=6.73, 2.04 Hz, 1H), 7.17 (tt, J=7.29, 5.65 Hz, 2H), 2.69 (s, 3H). ¹³C NMR (126 MHz, DMSO-d₆) δ 184.7-184.3 (m), 149.0, 135.8, 126.0, 123.1, 122.3, 120.4, 114.0, 111.8, 11.9. HRMS (ESI-TOF) m/z: [C₁₀H₈DNO+H]⁺ calcd for C₁₀H₉DNO: 161.0820, found: 161.0818.

### 2-Phenyl-1H-indole-3-carbaldehyde-d(3d-d)

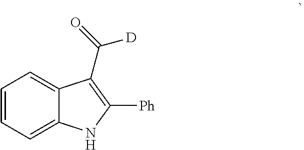 [SMILES: [2H]C(=O)c1c(-c2ccccc2)[nH]c2ccccc12]

The title product was prepared according to the general procedure and purified by column chromatography on silica gel eluting with hexane/ethyl acetate (6:1) to isolate as pale red soild about 15 mg (87%). ¹H NMR (500 MHz, DMSO) δ 12.41 (s, 1H), 9.99 (s, 0.01H), 8.24 (d, J=7.7 Hz, 1H), 7.84-7.75 (m, 2H), 7.66-7.56 (m, 3H), 7.52 (d, J=7.9 Hz, 1H), 7.34-7.22 (m, 2H). BC NMR (126 MHz, DMSO-d₆) δ 185.9-185.5 (m), 149.6, 136.4, 130.4 (2C), 130.3, 130.3, 129.5 (2C), 126.3, 124.2, 122.9, 121.6, 113.9, 112.5. HRMS (ESI-TOF) m/z: [C₁₅H₁₀DNO+H]⁺ calcd for C₁₅H₁₁DNO: 223.0976, found: 223.0972.

### 5-Methoxy-1H-indole-3-carbaldehyde (3e-d)

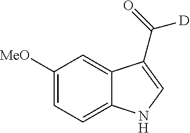 [SMILES: [2H]C(=O)c1c[nH]c2ccc(OC)cc12]

The title product was prepared according to the general procedure and purified by column chromatography on silica gel eluting with hexane/ethyl acetate (5:1) to isolate as white solid about 33 mg (54%). ¹H NMR (500 MHz, DMSO) δ 12.03 (s, 1H), 9.91 (s, 0.03H), 8.22 (s, 1H), 7.60 (d, J=2.55 Hz, 1H), 7.41 (d, J=8.82 Hz, 1H), 6.89 (dd, J=8.82, 2.56 Hz, 1H), 3.79 (s, 3H). ¹³C NMR (126 MHz, DMSO-d₆) δ 185.5-185.0 (m), 156.1, 138.9, 132.3, 125.4, 118.5, 113.8, 113.7, 103.0, 55.7. HRMS (ESI-TOF) m/z: [C₁₀H₈DNO₂+H]⁺ calcd for C₁₀H₉DNO₂: 177.0769, found: 177.0770.

### 5-(Benzyloxy)-1H-indole-3-carbaldehyde (3f-d)

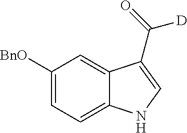 [SMILES: [2H]C(=O)c1c[nH]c2ccc(OCc3ccccc3)cc12]

The title product was prepared according to the general procedure and purified by column chromatography on silica gel eluting with hexane/ethyl acetate (5:1) to isolate as white solid about 18 mg (81%). ¹H NMR (500 MHz, DMSO-d₆) δ 9.80 (s, 0.03H), 8.13 (s, 1H), 7.61 (d, J=2.5 Hz, 1H), 7.43-7.37 (m, 2H), 7.36-7.28 (m, 3H), 7.27-7.20 (m, 1H), 6.88 (dd, J=8.8, 2.5 Hz, 1H), 5.04 (s, 2H). ¹³C NMR (126 MHz, DMSO-d₆) δ 185.4-185.0 (m), 155.1, 139.1, 137.9, 132.6, 128.8, 128.2 (2C), 125.4, 118.5, 114.2, 113.7, 104.5, 70.1. HRMS (ESI-TOF) m/z: [C₁₆H₁₂DNO₂+H]⁺ calcd for C₁₆H₁₃DNO₂: 253.1082, found: 253.1087.

### 6-(Benzyloxy)-1H-indole-3-carbaldehyde (3g-d)

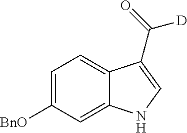 [SMILES: [2H]C(=O)c1c[nH]c2cc(OCc3ccccc3)ccc12]

The title product was prepared according to the general procedure and purified by column chromatography on silica gel eluting with hexane/ethyl acetate (5:1) to isolate as white solid about 36 mg (71%). ¹H NMR (500 MHz, DMSO) δ 11.94 (s, 1H), 9.88 (s, 0.03H), 8.16 (s, 1H), 7.97 (d, J=8.6 Hz, 1H), 7.53-7.45 (m, 2H), 7.43-7.37 (m, 2H), 7.36-7.25 (m, 1H), 7.08 (d, J=2.3 Hz, 1H), 6.96 (dd, J=8.6, 2.3 Hz, 1H), 5.15 (s, 2H). ¹³C NMR (126 MHz, DMSO-d₆) δ 185.4-185.0 (m), 156.2, 138.4 (2C), 137.7, 128.9, 128.2, 128.1, 121.9, 118.8, 112.9, 97.3, 70.0. HRMS (ESI-TOF) m/z: [C₁₆H₁₂DNO₂+H]⁺ calcd for C₁₆H₁₃DNO₂: 253.1082, found: 253.1085.

### 2-Vinyl-1H-indole-3-carbaldehyde (3h-d)

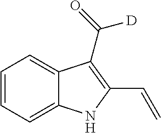

The title product was prepared according to the general procedure and purified by column chromatography on silica gel eluting with hexane/ethyl acetate (5:1) to isolate as white solid about 29 mg (85%). ¹H NMR (500 MHz, CDCl₃) δ 10.31 (s, 0.02H), 8.32-8.20 (m, 1H), 7.39-7.34 (m, 1H), 7.32-7.30 (m, 1H), 7.29-7.25 (m, 2H), 5.85 (d, J=17.7 Hz, 1H), 5.67 (d, J=11.3 Hz, 1H). ¹³C NMR (126 MHz, DMSO-d₆) δ 185.9-185.5 (m), 145.6, 136.7, 125.7, 124.8, 124.3, 123.0, 121.3, 121.1, 114.4, 112.2. HRMS (ESI-TOF) m/z: [C₁₁H₈DNO+H]⁺ calcd for C₁₁H₉DNO: 173.0820, found: 173.0824.

### 1-Benzyl-2-phenyl-1H-indole-3-carbaldehyde (3i-d)

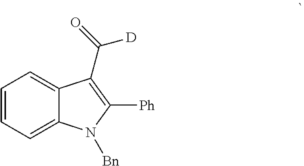 [SMILES: [2H]C(=O)c1c(-c2ccccc2)n(Cc2ccccc2)c2ccccc12]

The title product was prepared according to the general procedure and purified by column chromatography on silica gel eluting with hexane/ethyl acetate (5:1) to isolate as pale yellow oil about 47 mg (75%). ¹H NMR (500 MHz, CDCl₃) δ 9.75 (s, 0.02H), 8.46 (d, J=7.83 Hz, 1H), 7.51-7.39 (m, 5H), 7.35-7.31 (m, 1H), 7.27-7.21 (m, 6H), 6.95 (dd, J=7.53, 2.04 Hz, 2H), 5.27 (s, 2H). ¹³C NMR (126 MHz, CDCl₃) δ 187.0-186.6 (m), 151.9, 137.0, 136.4, 130.8, 130.1, 129.0, 128.7, 128.5 (2C) 126.0, 125.5, 124.3, 123.5, 122.3, 116.2, 110.9, 110.5, 110.0, 47.8. HRMS (ESI-TOF) m/z: [C₂₂H₁₆DNO+H]⁺ calcd for C₂₂H₁₇DNO: 313.1446, found: 313.1449.

### tert-Butyl 3-formyl-1H-indole-1-carboxylate (3j-d)

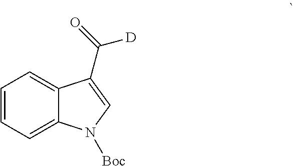 [SMILES: [2H]C(=O)c1cn(C(=O)OC(C)(C)C)c2ccccc12]

The title product was prepared according to the general procedure and purified by column chromatography on silica gel eluting with hexane/ethyl acetate (5:1) to isolate as white solid about 33 mg (68%). ¹H NMR (500 MHz, CDCl₃) δ 9.97 (s, 0.02H), 8.18-8.14 (m, 1H), 8.10 (s, 1H), 8.02 (d, J=8.22 Hz, 1H), 7.31-7.19 (m, 2H), 1.58 (s, 10H). ¹³C NMR (126 MHz, CDCl₃) δ 186.0-185.6 (m), 148.8, 136.5, 136.0, 126.1, 124.6, 122.2, 121.6, 115.2, 85.7, 28.1 (3C). HRMS (ESI-TOF) m/z: [C₁₄H₁₄DNO₃+H]⁺ calcd for C₁₄H₁₅DNO₃: 247.1187, found: 247.1190.

### 1-Butyl-1H-indole-3-carbaldehyde (3k-d)

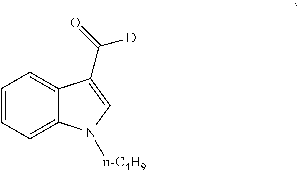

The title product was prepared according to the general procedure and purified by column chromatography on silica gel eluting with hexane/ethyl acetate (5:1) to isolate as white solid about 25 mg (61%). ¹H NMR (500 MHz, CDCl₃) δ 9.86 (s, 0.02H), 8.18 (dd, J=6.94, 2.06 Hz, 1H), 7.58 (s, 1H), 7.27-7.16 (m, 3H), 4.04 (t, J=7.17 Hz, 2H), 1.74 (p, J=7.30 Hz, 2H), 1.24 (dt, J=14.76, 7.43 Hz, 2H), 0.83 (t, J=7.28 Hz, 3H). ¹³C NMR (126 MHz, CDCl₃) δ 184.7-184.3 (m), 138.4, 137.2, 125.5, 123.9, 122.9, 122.1, 118.0, 110.1, 47.0, 31.8, 20.1, 13.6. HRMS (ESI-TOF) m/z: [C₁₃H₁₄DNO+H]⁺ calcd for C₁₃H₁₅DNO: 203.1289, found: 203.1283.

### 1-(4-Chlorobenzyl)-1H-indole-3-carbaldehyde-d (31-d)

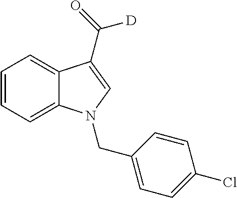 [SMILES: [2H]C(=O)c1cn(Cc2ccc(Cl)cc2)c2ccccc12]

The title product was prepared according to the general procedure and purified by column chromatography on silica gel eluting with hexane/ethyl acetate (5:1) to isolate as pale red soild about 22 mg (63%). ¹H NMR (500 MHz, CDCl₃) δ 9.93 (s, 0.05H), 8.31-8.22 (m, 1H), 7.63 (s, 1H), 7.31-7.16 (m, 5H), 7.09-6.99 (m, 2H), 5.26 (s, 2H). ¹³C NMR (126 MHz, CDCl₃) δ 184.4-184.2 (m), 138.5, 137.3, 134.2, 134.1, 129.1, 128.5, 125.5, 124.1, 123.0, 122.0, 118.4, 110.3, 53.7, 50.3, 45.4. HRMS (ESI-TOF) m/z: [C₁₆H₁₁DClNO+H]⁺ calcd for C₁₆H₁₂DClNO: 271.0743, found: 271.0736.

### 5-Fluoro-1H-indole-3-carbaldehyde-d (3m-d)

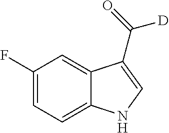 [SMILES: [2H]C(=O)c1c[nH]c2ccc(F)cc12]

The title product was prepared according to the general procedure and purified by column chromatography on silica gel eluting with hexane/ethyl acetate (5:1) to isolate as colorless oil about 21 mg (64%). ¹H NMR (500 MHz, DMSO) δ 12.23 (s, 1H), 9.93 (s, 0.04H), 8.36 (d, J=3.20 Hz, 1H), 7.77 (dd, J=9.55, 2.64 Hz, 1H), 7.53 (dd, J=8.84, 4.54 Hz, 1H), 7.12 (td, J=9.19, 2.66 Hz, 1H). ¹³C NMR (126 MHz, DMSO-d₆) δ 185.5-185.2 (m), 159.2 (d, J=239.4 Hz), 140.1, 134.1, 125.2 (d, J=11.3 Hz), 118.5, 114.2 (d, J=10.1 Hz), 112.1 (d, J=25.2 Hz), 106.2 (d, J=25.2 Hz). ¹⁹F NMR (470 MHz, DMSO) δ-121.2. HRMS (ESI-TOF) m/z: [C₉H₅DFNO+H]⁺ calcd for C₉H₆DFNO: 165.0569, found: 165.0568.

### 5-Bromo-1H-indole-3-carbaldehyde-d(3n-d)

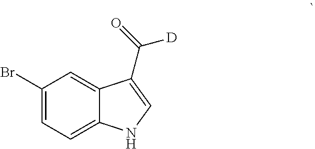 [SMILES: [2H]C(=O)c1c[nH]c2ccc(Br)cc12]

The title product was prepared according to the general procedure and purified by column chromatography on silica gel eluting with hexane/ethyl acetate (5:1) to isolate as colorless oil about 11 mg (71%). ¹H NMR (500 MHz, DMSO) δ 12.30 (s, 1H), 9.94 (s, 0.01H), 8.35 (d, J=3.03 Hz, 1H), 8.23 (d, J=1.93 Hz, 1H), 7.50 (d, J=8.60 Hz, 1H), 7.40 (dd, J=8.66, 2.03 Hz, 1H). ¹³C NMR (126 MHz, DMSO-d₆) δ 185.6-185.2 (m), 139.7, 136.2, 126.5, 126.4, 123.4, 117.8, 115.3, 115.0. HRMS (ESI-TOF) m/z: [C₉H₅DFNO+H]⁺ calcd for C₉H₆DBrNO: 224.9768, found: 224.9767.

### 5-Bromo-1-butyl-1H-indole-3-carbaldehyde-d (3o-d)

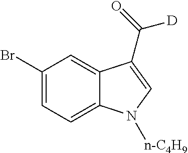

The title product was prepared according to the general procedure and purified by column chromatography on silica gel eluting with hexane/ethyl acetate (10:1) to isolate as white soild about 23 mg (58%). ¹H NMR (500 MHz, DMSO) δ 9.90 (s, 0.02H), 8.40 (s, 1H), 8.25 (d, J=2.02 Hz, 1H), 7.65 (d, J=8.77 Hz, 1H), 7.45 (dd, J=8.74, 2.03 Hz, 1H), 4.28 (t, J=7.09 Hz, 2H), 1.78 (p, J=7.21 Hz, 2H), 1.26 (h, J=7.30 Hz, 3H), 0.89 (t, J=7.39 Hz, 4H). ¹³C NMR (126 MHz, DMSO-d₆) δ 185.1-184.8 (m), 141.8, 136.3, 126.8, 126.5, 123.6, 116.7, 115.7, 113.8, 46.7, 31.8, 19.8, 13.9. HRMS (ESI-TOF) m/z: [C₁₃H₁₃DBrNO+H]⁺ calcd for C₁₃H₁₄DBrNO: 281.0394, found: 281.0394.

### 6-Chloro-1H-indole-3-carbaldehyde-d (3p-d)

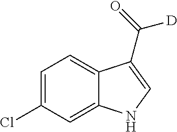 [SMILES: [2H]C(=O)c1c[nH]c2cc(Cl)ccc12]

The title product was prepared according to the general procedure and purified by column chromatography on silica gel eluting with hexane/ethyl acetate (5:1) to isolate as white soild about 18 mg (70%). ¹H NMR (500 MHz, DMSO) δ 12.21 (s, 1H), 9.94 (s, 0.02H), 8.34 (d, J=3.04 Hz, 1H), 8.08 (d, J=8.39 Hz, 1H), 7.58 (d, J=1.91 Hz, 1H), 7.25 (dd, J=8.43, 1.94 Hz, 1H). ¹³C NMR (126 MHz, DMSO-d₆) δ 185.6-185.1 (m), 139.7, 138.0, 128.4, 123.4, 122.9, 122.6, 118.3, 112.7. HRMS (ESI-TOF) m/z: [C₉H₅DClNO+H]⁺ calcd for C₉H₆DClNO: 181.0273, found: 181.0275.

### 7-Chloro-1H-indole-3-carbaldehyde-d (3q-d)

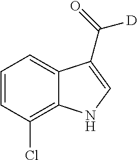 [SMILES: [2H]C(=O)c1c[nH]c2c(Cl)cccc12]

The title product was prepared according to the general procedure and purified by column chromatography on silica gel eluting with hexane/ethyl acetate (8:1) to isolate as white soild about 14 mg (64%). ¹H NMR (500 MHz, DMSO) δ 12.53 (s, 1H), 9.98 (s, 0.03H), 8.39 (d, J=3.18 Hz, 1H), 8.07 (d, J=7.84 Hz, 1H), 7.36 (d, J=7.68 Hz, 1H), 7.24 (t, J=7.79 Hz, 1H). ¹³C NMR (126 MHz, DMSO-d₆) δ 185.8-185.6 (m), 185.4, 139.6, 134.4, 126.5, 123.8, 123.5, 120.3, 119.3, 117.1. HRMS (ESI-TOF) m/z: [C₉H₅DClNO+H]⁺ calcd for C₉H₆DClNO: 181.0273, found: 181.0275.

### 3-(Formyl-d)-1H-indole-5-carbonitrile (3r-d)

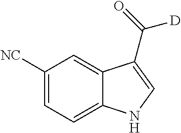 [SMILES: [2H]C(=O)c1c[nH]c2ccc(C#N)cc12]

The title product was prepared according to the general procedure and purified by column chromatography on silica gel eluting with hexane/ethyl acetate (5:1) to isolate as colorless oil about 18 mg (61%). ¹H NMR (500 MHz, DMSO) δ 12.58 (s, 1H), 10.00 (s, 0.01H), 8.51 (s, 1H), 8.47 (d, J=1.55 Hz, 1H), 7.71 (d, J=8.47 Hz, 1H), 7.64 (dd, J=8.48, 1.62 Hz, 1H). ¹³C NMR (126 MHz, DMSO-d₆) δ 185.8-185.4 (m), 140.7, 139.3, 126.9, 126.2, 124.5, 120.4, 118.4, 114.4, 104.9. HRMS (ESI-TOF) m/z: [C₁₀H₅DN₂O+H]⁺ calcd for C₁₀H₆DN₂O: 172.0616, found: 172.0615.

### Methyl 3-(formyl-d)-1H-indole-6-carboxylate (3s-d)

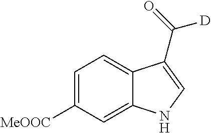 [SMILES: [2H]C(=O)c1c[nH]c2cc(C(=O)OC)ccc12]

The title product was prepared according to the general procedure and purified by column chromatography on silica gel eluting with hexane/ethyl acetate (3:1) to isolate as colorless oil about 25 mg (76%). ¹H NMR (500 MHz, DMSO) δ 12.44 (s, 1H), 9.99 (s, 0.01H), 8.50 (d, J=3.03 Hz, 1H), 8.19 (d, J=8.32 Hz, 1H), 8.15 (d, J=1.45 Hz, 1H), 7.84 (dd, J=8.36, 1.48 Hz, 1H). ¹³C NMR (126 MHz, DMSO-d₆) δ 185.7-185.3 (m), 167.1, 141.3, 136.9, 128.2, 125.0, 123.2, 121.1, 118.4, 114.6, 52.5. HRMS (ESI-TOF) m/z: [C₁₁H₈DNO₃+H]⁺ calcd for C₁₁H₉DNO₃: 205.0718, found: 205.0720.

### 1-Methyl-5-nitro-1H-indole-3-carbaldehyde-d (3t-d)

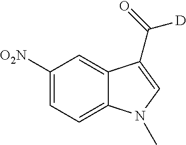 [SMILES: [2H]C(=O)c1cn(C)c2ccc([N+](=O)[O-])cc12]

The title product was prepared according to the general procedure and purified by column chromatography on silica gel eluting with hexane/ethyl acetate (3:1) to isolate as colorless oil about 23 mg (60%). ¹H NMR (500 MHz, CDCl₃) δ 10.02 (s, 0.05H), 9.16 (d, J=2.2 Hz, 1H), 8.21 (dd, J=9.0, 2.3 Hz, 1H), 7.83 (s, 1H), 7.41 (d, J=9.0 Hz, 1H), 3.94 (s, 3H). ¹³C NMR (126 MHz, CDCl₃) δ 184.2-183.8 (m), 144.1, 141.2, 140.4, 124.7, 119.6, 119.3, 119.0, 110.1, 34.2. HRMS (ESI-TOF) m/z: [C₁₀H₇DN₂O₃+H]⁺ calcd for C₁₀H₈DN₂O₃: 206.0670, found: 206.0668.

### 1H-Indole-3,4-dicarbaldehyde (3u-d)

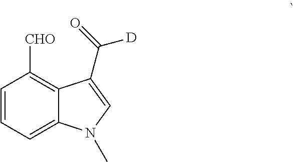

The title product was prepared according to the general procedure and purified by column chromatography on silica gel eluting with hexane/ethyl acetate (5:1) to isolate as white solid about 24 mg (65%). ¹H NMR (500 MHz, CDCl₃) δ 11.02 (s, 1H), δ 10.19 (s, 0.04H), 7.96-7.88 (m, 2H), 7.61 (dd, J=8.1, 1.1 Hz, 1H), 7.46 (t, J=7.8 Hz, 1H), 3.93 (s, 3H). ¹³C NMR (126 MHz, CDCl₃) δ 195.0, 185.5-185.1 (m), 142.2, 139.3, 130.5, 125.5, 123.5, 123.4, 119.1, 116.0, 34.1. HRMS (ESI-TOF) m/z: [C₁₁H₈DNO₂+H]⁺ calcd for C₁₁H₉DNO₂: 189.0769, found: 189.0768.

### 1-Methyl-2-phenyl-1H-indole-3-carbaldehyde-d (3v-d)

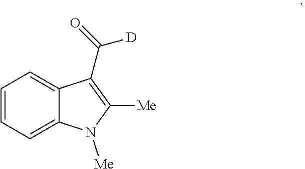 [SMILES: [2H]C(=O)c1c(C)n(C)c2ccccc12]

The title product was prepared according to the general procedure and purified by column chromatography on silica gel eluting with hexane/ethyl acetate (6:1) to isolate as pale yellow soild about 36 mg (91%). ¹H NMR (500 MHz, DMSO) δ 9.53 (s, 0.03H), 8.18-8.11 (m, 1H), 7.61-7.50 (m, 6H), 7.33-7.19 (m, 2H), 3.60 (s, 3H). ¹³C NMR (126 MHz, DMSO-d₆) δ 185.4-185.2 (m), 151.7, 137.6, 131.5 (2C), 130.4, 129.1 (2C), 128.6, 125.0, 124.2, 123.4, 121.4, 114.8, 111.5, 31.5. HRMS (ESI-TOF) m/z: [C₁₆H₁₂DNO+H]⁺ calcd for C₁₆H₁₃DNO: 237.1133, found: 237.1130.

### 2-((3-(Formyl-d)-1-methyl-1H-indol-6-yl)amino)-2-oxoethyl-2-(2-((2,6-dichlorophenyl)amino)phenyl)acetate (3w-d)

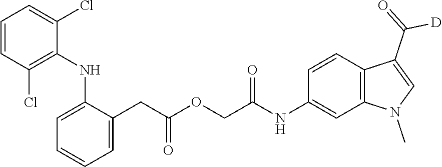 [SMILES: [2H]C(=O)c1cn(C)c2cc(NC(=O)COC(=O)Cc3ccccc3Nc3c(Cl)cccc3Cl)ccc12]

The title product was prepared according to the general procedure and purified by column chromatography on silica gel eluting with hexane/ethyl acetate (3:1) to isolate as pale red soild about 18 mg (45%). ¹H NMR (500 MHz, CDCl₃) δ 9.95 (s, 0.05H), 8.22 (d, J=8.14 Hz, 1H), 7.66 (s, 1H), 7.33-7.25 (m, 4H), 7.02-6.82 (m, 4H), 6.63 (d, J=23.57 Hz, 1H), 6.51 (d, J=7.84 Hz, 1H), 5.30 (s, 2H), 4.71 (s, 2H), 3.91 (d, J=5.46 Hz, 3H), 3.80 (s, 3H). ¹³C NMR (126 MHz, CDCl₃) δ 184.3-184.1 (m), 171.51, 171.49, 167.5, 142.8, 141.5, 139.7, 137.84, 137.80, 137.6, 131.0, 130.9, 130.0, 129.55, 129.51, 129.0, 128.9, 128.6, 128.19, 128.16, 126.2, 125.5, 125.4, 124.13, 124.09, 124.0, 123.9, 123.5, 123.1, 122.24, 122.22, 122.20, 120.8, 120.7, 118.51, 118.49, 117.9, 111.0, 110.3, 110.2, 77.3, 67.7, 61.39, 61.36w, 38.1, 33.8. HRMS (ESI-TOF) m/z: [C₂₆H₂₀DCl₂N₃O₄+H]⁺ calcd for C₂₆H₂₁DCl₂N₃O₄: 511.1045, found: 511.1044.

### (3-(Formyl-d)-1-methyl-1H-indol-6-yl)methyl 2-(4-isobutylphenyl)propanoate (3x-d)

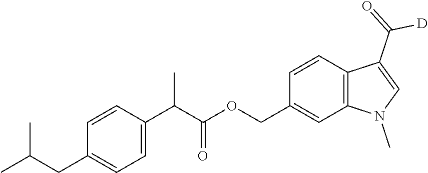 [SMILES: [2H]C(=O)c1cn(C)c2cc(COC(=O)C(C)c3ccc(CC(C)C)cc3)ccc12]

The title product was prepared according to the general procedure and purified by column chromatography on silica gel eluting with hexane/ethyl acetate (7:1) to isolate as white soild about 21 mg (51%). ¹H NMR (500 MHz, CDCl₃) δ 9.98 (s, 0.02H), 8.29-8.18 (m, 1H), 7.66 (s, 1H), 7.27-7.23 (m, 3H), 7.17-7.09 (m, 2H), 5.28 (s, 2H), 3.79 (s, 4H), 2.48 (d, J=7.17 Hz, 2H), 1.87 (dq, J=13.52, 6.77 Hz, 1H), 1.55 (d, J=7.17 Hz, 3H), 0.93 (d, J=6.58 Hz, 6H). ¹³C NMR (126 MHz, CDCl₃) δ 184.4-183.9 (m), 174.6, 140.6, 139.6, 137.8, 137.7, 132.2, 129.3, 127.3, 125.0, 123.0, 122.1, 117.9, 109.5, 66.7, 45.2, 45.0, 33.6, 30.2, 22.4, 18.5. HRMS (ESI-TOF) m/z: [C₂₄H₂₆DNO₃+H]⁺ calcd for C₂₄H₂₇DNO₃: 379.2126, found: 379.2119.

### 3-(Formyl-d)-1H-indol-4-yl)methyl (2S,5R)-3,3-dimethyl-7-oxo-4-thia-1-azabicyclo[3.2.0]heptane-2-carboxylate 4,4-dioxide (3y-d)

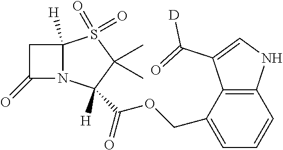 [SMILES: [2H]C(=O)c1c[nH]c2cccc(COC(=O)[C@@H]3N4C(=O)C[C@H]4S(=O)(=O)C3(C)C)c12]

The title product was prepared according to the general procedure and purified by column chromatography on silica gel eluting with hexane/ethyl acetate (6:1) to isolate as pale yellow soild about 35 mg (57%). ¹ 1H NMR (500 MHz, CDCl₃) δ 9.92 (s, 0.04H), δ 7.94 (d, J=3.1 Hz, 1H), 7.46 (dd, J=6.2, 2.9 Hz, 1H), 7.36-7.29 (m, 2H), 6.03 (d, J=11.5 Hz, 1H), 5.80 (d, J=11.5 Hz, 1H), 4.56 (dd, J=4.2, 2.2 Hz, 1H), 4.33 (s, 1H), 3.48-3.29 (m, 2H), 1.44 (s, 3H), 1.36 (s, 3H). ¹³C NMR (126 MHz, CDCl₃) δ 184.1-183.7 (m), 170.8, 166.8, 138.9, 137.9, 128.5, 125.7, 124.6, 123.0, 120.6, 112.8, 99.9, 63.3, 61.0, 38.1, 20.0, 18.2. HRMS (ESI-TOF) m/z: [C₁₈H₁₇DN₂O₆S+H]⁺ calcd for C₁₈H₁₈DN₂O₆S: 392.1021, found: 392.1025.

### (3-(Formyl-d)-1-methyl-1H-indol-6-yl)methyl 2-(1-(4-chlorobenzoyl)-5-methoxy-2-methyl-1H-indol-3-yl)acetate (3z-d)

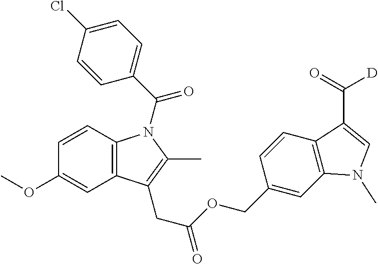

The title product was prepared according to the general procedure and purified by column chromatography on silica gel eluting with hexane/ethyl acetate (2:1) to isolate as white soild about 29 mg (63%). ¹H NMR (500 MHz, CDCl₃) δ 9.84 (s, 0.04H), 8.23 (dd, J=8.1, 0.7 Hz, 1H), 7.59 (dq, J=8.5, 2.0 Hz, 2H), 7.45-7.37 (m, 2H), 7.27-7.20 (m, 2H), 6.95-6.76 (m, 2H), 6.63 (ddd, J=9.0, 6.4, 2.5 Hz, 1H), 5.26 (s, 2H), 3.73 (s, 3H), 3.67 (d, J=10.2 Hz, 3H), 2.32 (d, J=4.1 Hz, 3H). ¹³C NMR (126 MHz, CDCl₃) δ 184.5-184.3 (m), 170.7, 168.3, 156.0, 139.9, 139.3, 137.8, 135.9, 133.8, 131.8, 131.1, 130.8, 130.6, 129.15, 129.111, 125.2, 123.3, 122.2, 115.0, 112.5, 111.7, 109.9, 101.4, 67.2, 55.6, 55.6, 33.6, 30.6, 13.5, 13.4. HRMS (ESI-TOF) m/z: [C₃₀H₂₄DClN₂O₅+H]⁺ calcd for C₃₀H₂₅DClN₂O₅: 530.1588, found: 530.1581.

### (3R,5S,7S,8R,9S,10S,13R,14S,17R)-17-((R)-5-((3-(Formyl-d)-1H-indol-4-yl)methoxy)-5-oxopentan-2-yl)-10,13-dimethylhexadecahydro-1H-cyclopenta[a]phenanthrene-3,7-diyl diacetate (3aa-d)

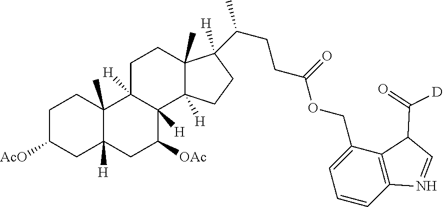 [SMILES: [2H]C(=O)C1C=Nc2cccc(COC(=O)CC[C@@H](C)[C@H]3CC[C@H]4[C@@H]5[C@@H](OC(C)=O)C[C@@H]6C[C@H](OC(C)=O)CC[C@]6(C)[C@H]5CC[C@]34C)c21]

The title product was prepared according to the general procedure and purified by column chromatography on silica gel eluting with hexane/ethyl acetate (2:1) to isolate as white soild about 30 mg (42%). ¹H NMR (500 MHz, CDCl₃) δ 10.01 (s, 0.04H) δ 8.94 (s, 1H), 7.93 (d, J=3.1 Hz, 1H), 7.41 (dd, J=6.9, 2.3 Hz, 1H), 7.32-7.26 (m, 2H), 5.72 (s, 2H), 4.78-4.60 (m, 2H), 2.36 (ddd, J=14.5, 9.6, 4.9 Hz, 2H), 2.23 (ddd, J=14.9, 8.7, 6.4 Hz, 2H), 2.01 (s, 3H), 1.96 (s, 3H), 1.83-1.40 (m, 25H), 1.35-1.10 (m, 18H), 0.94 (s, 4H), 0.88-0.82 (m, 5H), 0.61 (s, 3H). ¹³C NMR (126 MHz, CDCl₃) δ 184.6-184.2 (m), 174.0, 136.4, 124.2 (2C), 122.9, 112.1, 73.6, 66.3, 55.2, 54.9, 43.5, 42.1, 39.9 (2C), 39.4, 35.2, 34.5, 34.0, 32.9, 31.2, 30.8, 28.4, 26.4, 25.6, 23.2, 21.9, 21.4, 21.2, 18.3, 12.0. HRMS (ESI-TOF) m/z: [C₃₈H₅₀DNO₇+H]⁺ calcd for C₃₈H₅₀DNO₇: 635.3801, found: 635.3806.

### (3-(Formyl-d)-1H-indol-6-yl)methyl 2-(2-fluoro-[1,1′-biphenyl]-4-yl)propanoate (3ab-d)

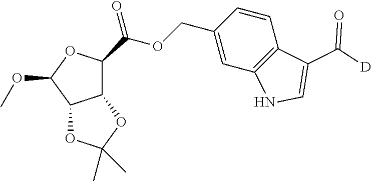 [SMILES: [2H]C(=O)c1c[nH]c2cc(COC(=O)[C@@H]3O[C@H](OC)[C@H]4OC(C)(C)O[C@H]43)ccc12]

The title product was prepared according to the general procedure and purified by column chromatography on silica gel eluting with hexane/ethyl acetate (2:1) to isolate as white soild about 28 mg (53%). ¹H NMR (500 MHz, DMSO-d₆) δ 9.93 (s, 0.04H), δ 8.31 (d, J=3.1 Hz, 1H), 8.05 (d, J=8.1 Hz, 1H), 7.58-7.37 (m, 7H), 7.31-7.13 (m, 3H), 5.24 (q, J=12.2 Hz, 2H), 1.46 (d, J=7.1 Hz, 3H). ¹³C NMR (126 MHz, DMSO-d₆) δ 185.6-185.2 (m), 173.7, 170.8, 159.4 (d, J=239.4 Hz), 142.85, 142.78, 139.5, 137.4, 135.3, 131.7, 131.3 (d, J=3.8 Hz), 129.2, 129.2 (d, J=2.5 Hz) 128.3, 127.4 (d, J=13.9 Hz), 124.5, 124.4, 123.0, 121.2, 118.5, 115.7 (d, J=23.9 Hz), 112.8, 67.0, 60.2, 49.1, 44.4, 21.2, 18.8, 14.6. ¹⁹F NMR (470 MHz, DMSO-d₆)-118.31. HRMS (ESI-TOF) m/z: [C₂₅H₁₉DFNO₃+H]⁺ calcd for C₂₅H₂₀DFNO₃: 403.1563, found: 403.1564.

### 3-(Formyl-d)-1H-indol-6-yl)methyl (3aR,4R,6S,6aS)-6-methoxy-2,2-dimethyltetrahydrofuro[3,4-d][1,3]dioxole-4-carboxylate (3ac-d)

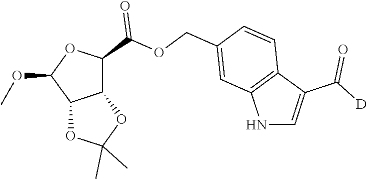 [SMILES: [2H]C(=O)c1c[nH]c2cc(COC(=O)[C@@H]3O[C@H](OC)[C@H]4OC(C)(C)O[C@H]43)ccc12]

The title product was prepared according to the general procedure and purified by column chromatography on silica gel eluting with hexane/ethyl acetate (2:1) to isolate as white soild about 28 mg (58%). ¹H NMR (500 MHz, CDCl₃) δ 9.92 (s, 0.05H), δ 8.19 (d, J=8.1 Hz, 1H), 7.79 (s, 1H), 7.43 (s, 1H), 7.23 (d, J=8.1 Hz, 1H), 5.25 (d, J=12.0 Hz, 1H), 5.19-5.13 (m, 2H), 4.93 (s, 1H), 4.56 (s, 1H), 4.45 (d, J=5.8 Hz, 1H), 3.14 (s, 3H), 1.38 (s, 3H), 1.22 (s, 3H). ¹³C NMR (126 MHz, CDCl₃) δ 185.7-185.3 (m), 170.3, 137.1, 136.9, 131.4, 124.6, 123.8, 122.0, 119.2, 112.8, 112.5, 109.4, 84.3, 83.8, 82.0, 67.7, 55.5, 26.4, 25.0. RMS (ESI-TOF) m/z: [C₁₉H₂₀DNO₇+H]⁺ calcd for C₁₉H₂₁DNO₇: 377.1454, found: 377.1457.

### (3-(Formyl-d)-1H-indol-6-yl)methyl (3aR,5S,5aR,8aS,8bR)-2,2,7,7-tetramethyltetrahydro-5H-bis([1,3]dioxolo)[4,5-b:4′,5′-d]pyran-5-carboxylate (3ad-d)

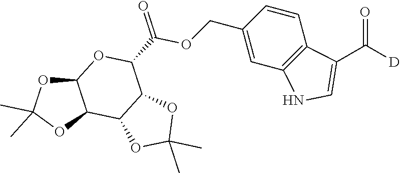 [SMILES: [2H]C(=O)c1c[nH]c2cc(COC(=O)[C@H]3O[C@@H]4OC(C)(C)O[C@@H]4[C@H]4OC(C)(C)O[C@H]43)ccc12]

The title product was prepared according to the general procedure and purified by column chromatography on silica gel eluting with hexane/ethyl acetate (2:1) to isolate as white soild about 25 mg (55%). ¹H NMR (500 MHz, CDCl₃) δ 9.97 (s, 0.05H), δ 8.24 (d, J=8.1 Hz, 1H), 7.81 (d, J=3.2 Hz, 1H), 7.62 (s, 1H), 7.30 (d, J=8.2 Hz, 1H), 5.65 (d, J=4.9 Hz, 1H), 5.51 (d, J=11.9 Hz, 1H), 5.24 (d, J=11.9 Hz, 1H), 4.68-4.55 (m, 2H), 4.46 (s, 1H), 4.37 (dd, J=5.0, 2.7 Hz, 1H), 1.47 (s, 3H), 1.36-1.24 (m, 9H). ¹³C NMR (126 MHz, CDCl₃) δ 185.5-185.0 (m), 168.9, 136.9, 131.1, 124.7, 123.8, 121.8, 119.1, 113.4, 110.1, 109.3, 96.5, 72.0, 70.6, 70.3, 68.7, 68.0, 26.0 (2C), 24.8 (2C). HRMS (ESI-TOF) m/z: [C₂₂H₂₄DNO₈+H]⁺ calcd for C₂₂H₂₅DNO₈: 433.1716, found: 433.1715.

### (3-(Formyl-d)-1H-indol-6-yl)methyl(tert-butoxycarbonyl)-L-phenylalanyl-L-alaninate (3ae-d)

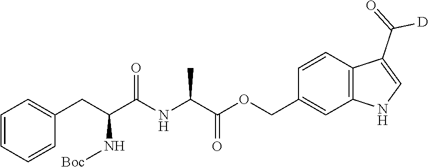 [SMILES: [2H]C(=O)c1c[nH]c2cc(COC(=O)[C@H](C)NC(=O)[C@H](Cc3ccccc3)NC(=O)OC(C)(C)C)ccc12]

The title product was prepared according to the general procedure and purified by column chromatography on silica gel eluting with hexane/ethyl acetate (2:1) to isolate as white soild about 31 mg (47%). ¹H NMR (500 MHz, CDCl₃) δ 9.93 (s, 0.03H), δ 8.21 (t, J=9.2 Hz, 1H), 7.78 (d, J=10.1 Hz, 1H), 7.39-7.30 (m, 1H), 7.24-7.14 (m, 6H), 5.26-5.14 (m, 2H), 4.59-4.50 (m, 1H), 4.48-4.38 (m, 1H), 3.10-2.91 (m, 2H), 1.35 (s, 10H), 1.21 (dd, J=17.4, 6.6 Hz, 3H). ¹³C NMR (126 MHz, CDCl₃) δ 185.5-185.1 (m), 172.1, 171.1, 171.0, 155.6, 137.1, 136.7, 136.3, 131.5, 129.34, 129.29, 128.7, 127.1, 124.4, 122.7, 121.9, 119.2, 111.4, 80.5, 67.3, 60.4, 55.8, 39.0, 28.3 (3C), 17.8. HRMS (ESI-TOF) m/z: [C₂₇H₃₀DN₃O₆+H]⁺ calcd for C₂₇H₃₁DN₃O₆: 495.2348, found: 495.2345.

### 5-Bromo-1-methyl-1H-indole-3-carbaldehyde (3x)

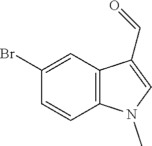

The title product was prepared according to the general procedure 2 and purified by column chromatography on silica gel eluting with hexane/ethyl acetate (8:1) to isolate as red soild about 29 mg (61%). ¹H NMR (500 MHz, CDCl₃) δ 9.95 (s, 1H), 8.45 (d, J=1.8 Hz, 1H), 7.65 (s, 1H), 7.43 (dd, J=8.7, 1.9 Hz, 1H), 7.21 (d, J=8.7 Hz, 1H), 3.85 (s, 3H). ¹³C NMR (126 MHz, CDCl₃) δ 184.1, 139.5, 136.6, 127.1, 126.8, 124.8, 117.5, 116.7, 111.3, 33.9.

### 1-Methyl-1H-indole-3-carbaldehyde (3w)

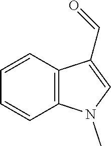

The title product was prepared according to the general procedure and purified by column chromatography on silica gel eluting with hexane/ethyl acetate (7:1) to isolate as white soild about 27 mg (85%). ¹H NMR (500 MHz, CDCl₃) δ 9.88 (d, J=2.3 Hz, 1H), 8.24-8.11 (m, 1H), 7.56 (d, J=2.9 Hz, 1H), 7.27-7.17 (m, 3H), 3.75 (s, 3H). ¹³C NMR (126 MHz, CDCl₃) δ 184.4, 139.1, 137.9, 125.3, 124.0, 123.0, 122.1, 118.1, 109.8, 33.7.

### 5-Bromo-1-methylindoline-2,3-dione (5x)

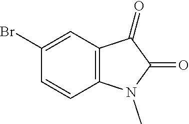

The title product was prepared according to the general procedure 2 and purified by column chromatography on silica gel eluting with hexane/ethyl acetate (4:1) to isolate as red soild about 15 mg (32%). ¹H NMR (500 MHz, CDCl₃) δ 7.77-7.64 (m, 2H), 6.79 (d, J=8.2 Hz, 1H), 3.23 (s, 3H). ¹³C NMR (126 MHz, CDCl₃) δ 182.2, 157.5, 150.1, 140.6, 128.1, 118.6, 116.7, 111.6, 26.4.

For reasons of completeness, various aspects of the technology are set out in the following clauses:

Clause 1. A method for preparing a deuterated compound of formula (I), or a salt thereof,

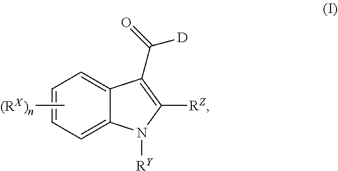

wherein:
R^{X} is halogen, C₁₋₄alkyl, C₁₋₂haloalkyl, R^{a}, —NO₂, —CN, —CO₂C₁₋₄alkyl, —C(O)C₁₋₄alkyl, —C(O)H, —OC₁₋₃alkylene-R^{a}, —C₁₋₃alkylene-R^{a}, -L¹-A¹, or -L²-A²;
R^{a} is C₃₋₄cycloalkyl, —OC₁₋₄alkyl, —OC₁₋₂haloalkyl, —OC₃₋₄cycloalkyl, or phenyl, wherein, at each occurrence, the phenyl is optionally substituted with 1-2 substituents selected from the group consisting of halogen, C₁₋₄alkyl, C₁₋₂haloalkyl, —NO₂, —CN, —OC₁₋₄alkyl, —OC₁₋₂haloalkyl, or —OC₃₋₄cycloalkyl;
R^{Y} is hydrogen, C₁₋₄alkyl, or an amine protecting group;
R^{Z} is hydrogen, C₁₋₄alkyl, C₂₋₄alkenyl, or unsubstituted phenyl;
L¹ is —C₁₋₃alkylene- or —OC₁₋₃alkylene-;
A¹ is a drug moiety;
   wherein A¹ is linked to L¹ by

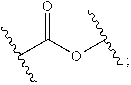 [SMILES: *OC(*)=O]

L² is —NH—;
A² is a drug moiety;
   wherein A² is linked to L² by:

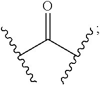 [SMILES: *C(*)=O]

and
n is 0, 1, or 2;
the method comprising:
   mixing D-CO₂D with a compound of formula (II),

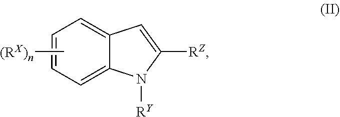

in an organic solvent to form a mixture; and
exposing the mixture of (i) to light, thereby producing the deuterated compound of formula (I), or a salt thereof.

Clause 2. The method of clause 1, wherein n is 0 or 1.

Clause 3. The method of clause 1 or 2, wherein R^{X} is halogen, C₁₋₄alkyl, —NO₂, —CN, —OC₁₋₄alkyl, —CO₂C₁₋₄alkyl, —C(O)H, -L¹-A¹, or -L²-A².

Clause 4. The method of any one of clauses 1-3, wherein L¹ is —CH₂—.

Clause 5. The method of any one of clauses 1-3, wherein the drug moiety at A¹ or A² is a non-steroidal anti-inflammatory drug (NSAID) moiety or a β-lactamase inhibitor moiety.

Clause 6. The method of any one of clauses 1-5, wherein Rₓ is selected from the group consisting of:

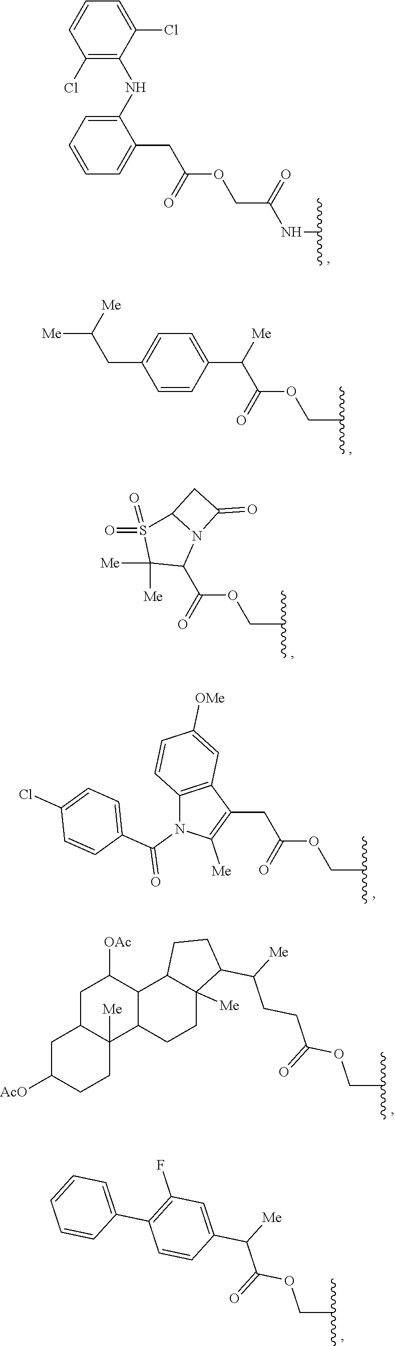

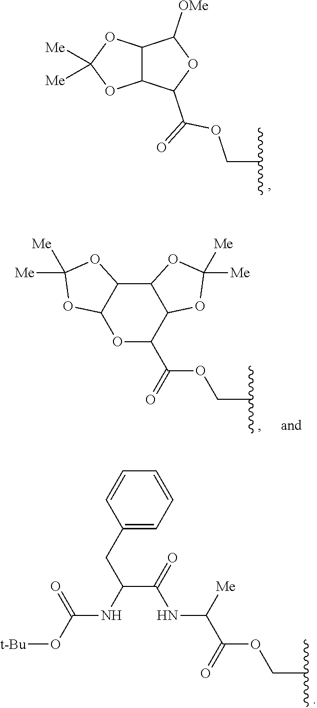 [SMILES: *COC(=O)C1OC(OC)C2OC(C)(C)OC12 ; *COC(=O)C1OC2OC(C)(C)OC2C2OC(C)(C)OC12 ; *COC(=O)C(C)NC(=O)C(Cc1ccccc1)NC(=O)OC(C)(C)C]

Clause 7. The method of any one of clauses 1-6, wherein R is hydrogen, C₁₋₄alkyl,

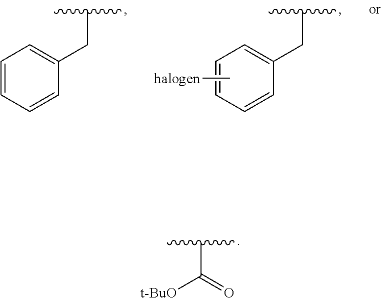

Clause 8. The method of any one of clauses 1-7, wherein R^{Z} is hydrogen, methyl,

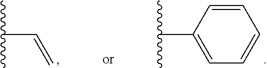

Clause 9. The method of any one of clauses 1-8, wherein the compound of formula (I) is selected from the group consisting of:

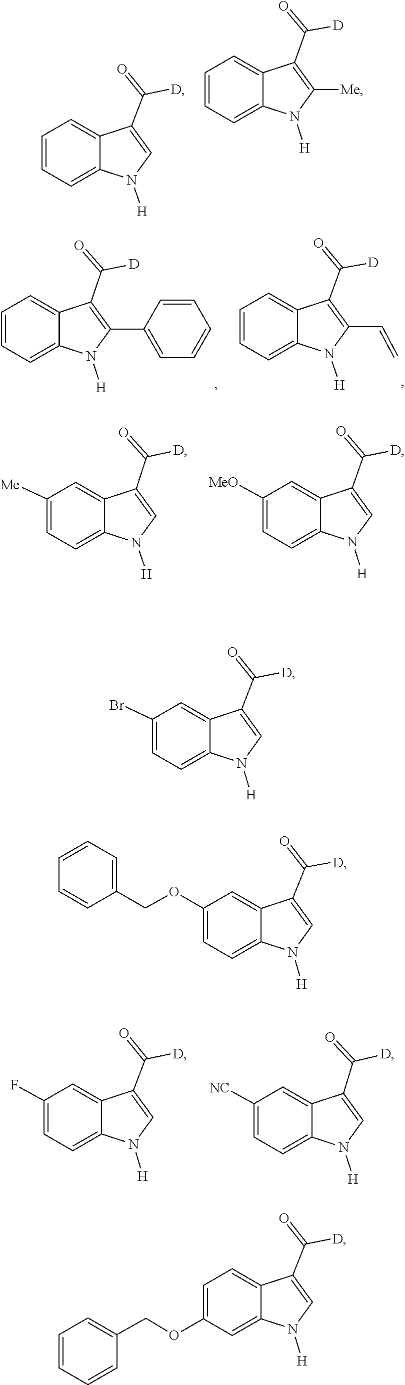

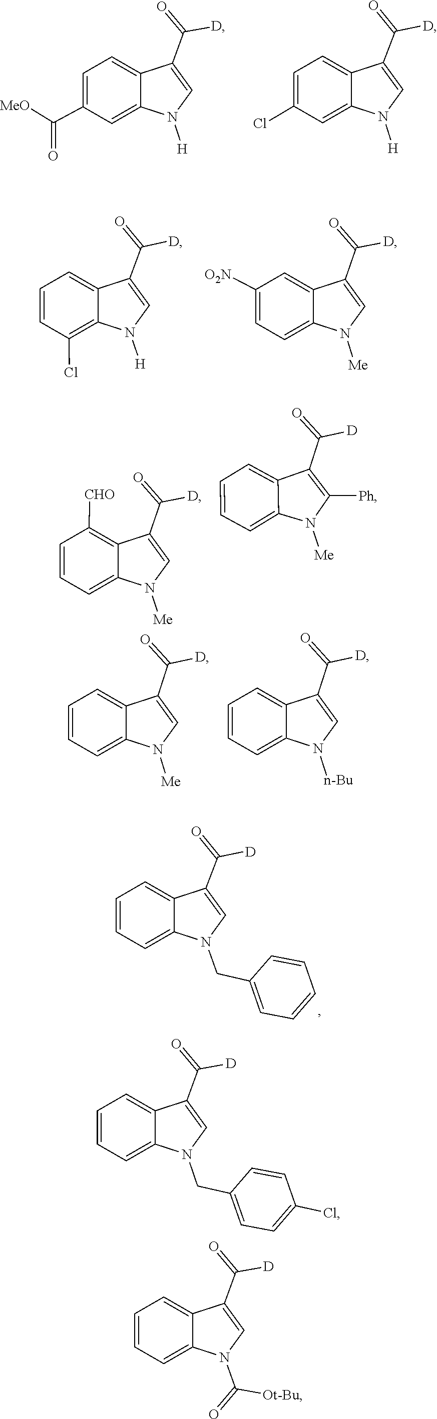

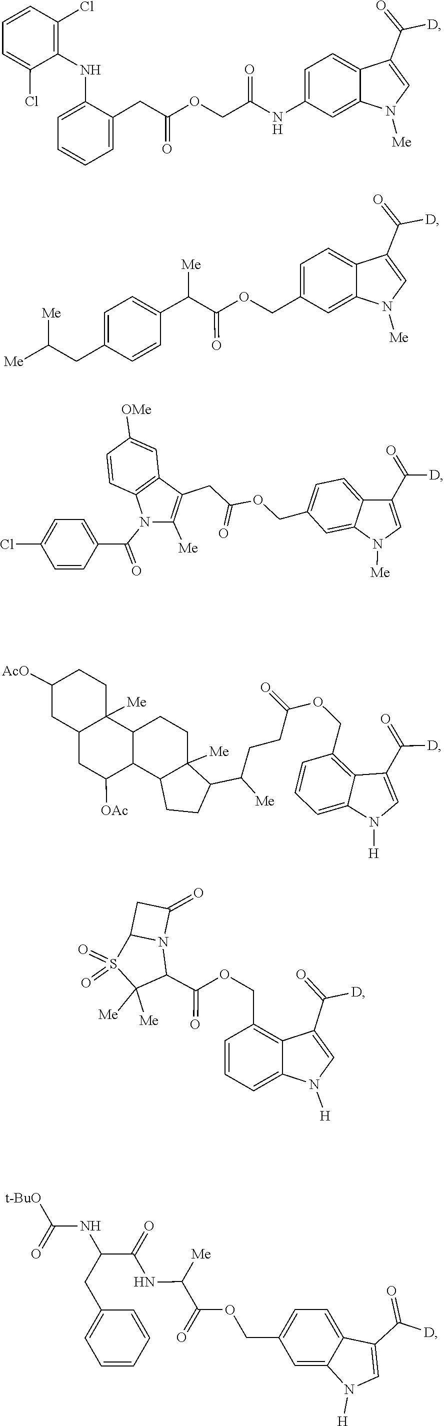

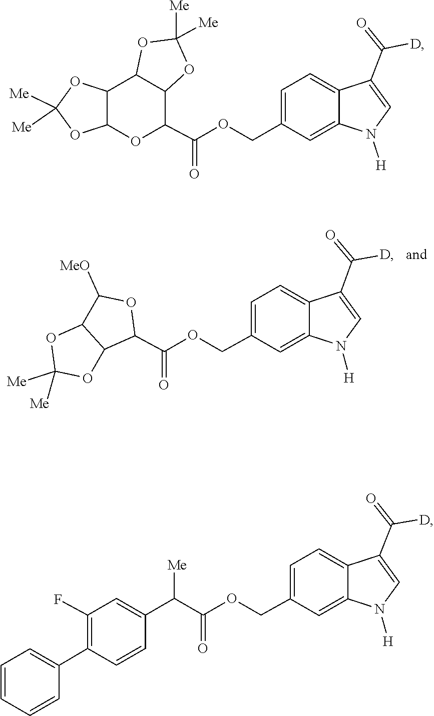

or a salt thereof.

Clause 10. The method of any one of clauses 1-9, wherein the exposing the mixture of (i) to light occurs in the presence of air.

Clause 11. The method of any one of clauses 1-10, wherein the mixture is essentially water (H₂O) free.

Clause 12. The method of any one of clauses 1-11, wherein the base is sodium acetate (NaOAc).

Clause 13. The method of any one of clauses 1-12, wherein the mixture is catalyst free. Clause 14. The method of any one of clauses 1-13, wherein the solvent is a polar aprotic solvent.

Clause 15. A deuterated compound of formula (I), or a salt thereof,

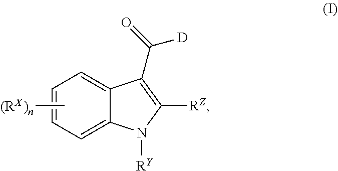

wherein:
R^{X} is halogen, C₁₋₄alkyl, C₁₋₂haloalkyl, R^{a}, —NO₂, —CN, —CO₂C₁₋₄alkyl, —C(O)C₁₋₄alkyl, —C(O)H, —OC₁₋₃alkylene-R^{a}, —C₁₋₃alkylene-R^{a}, -L¹-A¹, or -L²-A²;
R^{a} is C₃₋₄cycloalkyl, —OC₁₋₄alkyl, —OC₁₋₂haloalkyl, —OC₃₋₄cycloalkyl, or phenyl, wherein, at each occurrence, the phenyl is optionally substituted with 1-2 substituents selected from the group consisting of halogen, C₁₋₄alkyl, C₁₋₂haloalkyl, —NO₂, —CN, —OC₁₋₄alkyl, —OC₁₋₂haloalkyl, or —OC₃₋₄cycloalkyl;
R^{Y} is hydrogen, C₁₋₄alkyl, or an amine protecting group;
R^{Z} is hydrogen, C₁₋₄alkyl, C₂₋₄alkenyl, or unsubstituted phenyl;
L¹ is —C₁₋₃alkylene- or —OC₁₋₃alkylene-;
A¹ is a drug moiety;
   wherein A¹ is linked to L¹ by

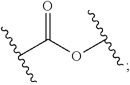 [SMILES: *OC(*)=O]

L² is —NH—;
A² is a drug moiety;
   wherein A² is linked to L² by:

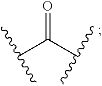 [SMILES: *C(*)=O]

and
n is 0, 1, or 2.

Clause 16. The compound of clause 15, or a salt thereof, wherein n is 0 or 1.

Clause 17. The compound of clause 15 or 16, or a salt thereof, wherein R^{X} is halogen, C₁₋₄alkyl, —NO₂, —CN, —OC₁₋₄alkyl, —CO₂C₁₋₄alkyl, —C(O)H, -L¹-A¹, or -L²-A²;

Clause 18. The compound of any one of clauses 15-17, or a salt thereof, wherein L¹ is —CH₂—.

Clause 19. The compound of any one of clauses 15-18, or a salt thereof, wherein the drug moiety at A¹ or A² is a non-steroidal anti-inflammatory drug (NSAID) moiety or a β-lactamase inhibitor moiety.

Clause 20. The compound of any one of clauses 15-19, or a salt thereof, wherein R^{X} is selected from the group consisting of

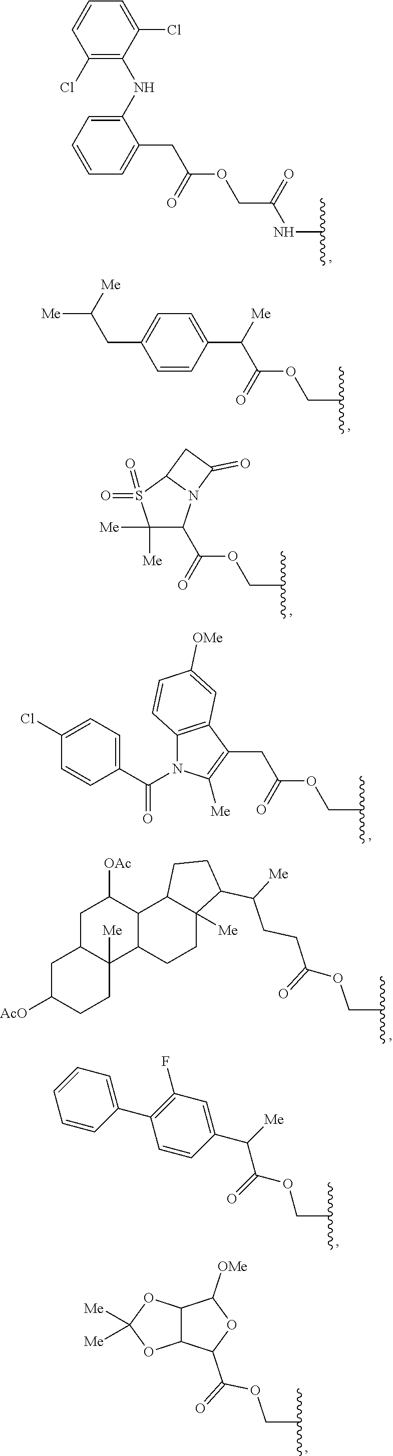

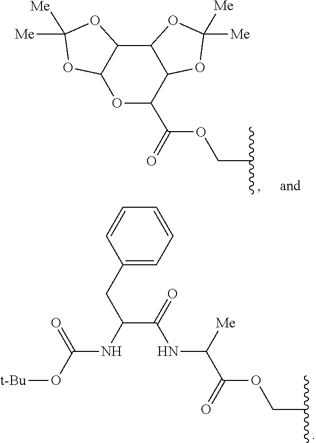

Clause 21. The compound of any one of clauses 15-20, or a salt thereof, wherein R is hydrogen, C₁₋₄alkyl,

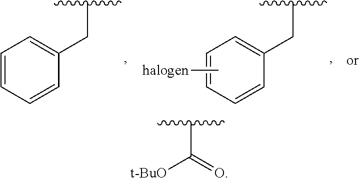

Clause 22. The compound of any one of clauses 15-21, or a salt thereof, wherein R^{Z} is hydrogen, methyl,

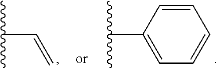

Clause 23. The compound of any one of clauses 15-22, selected from the group consisting of:

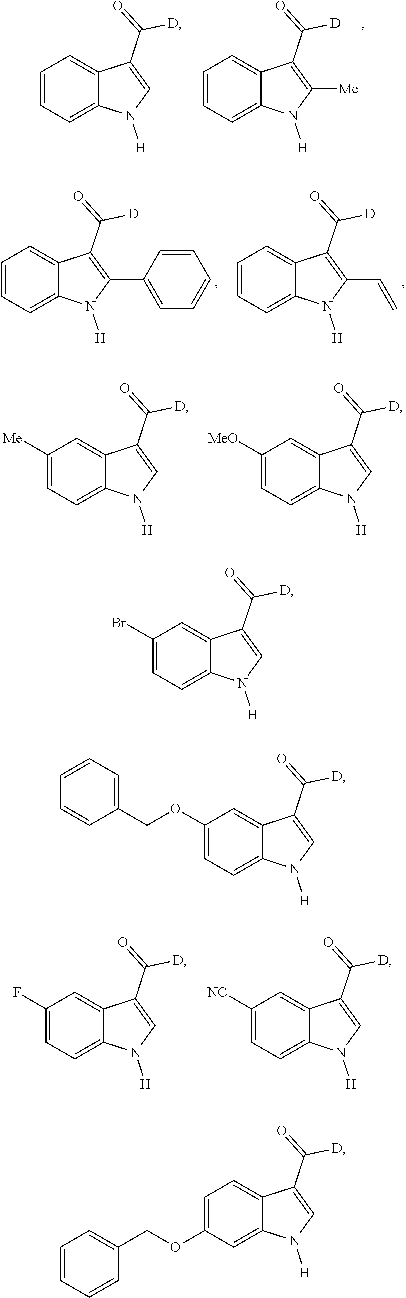

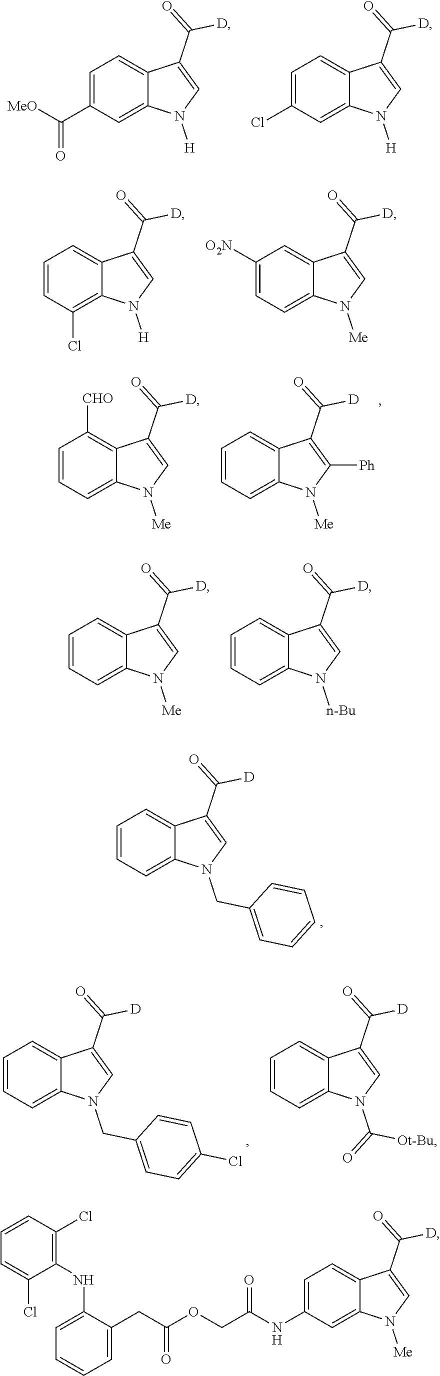

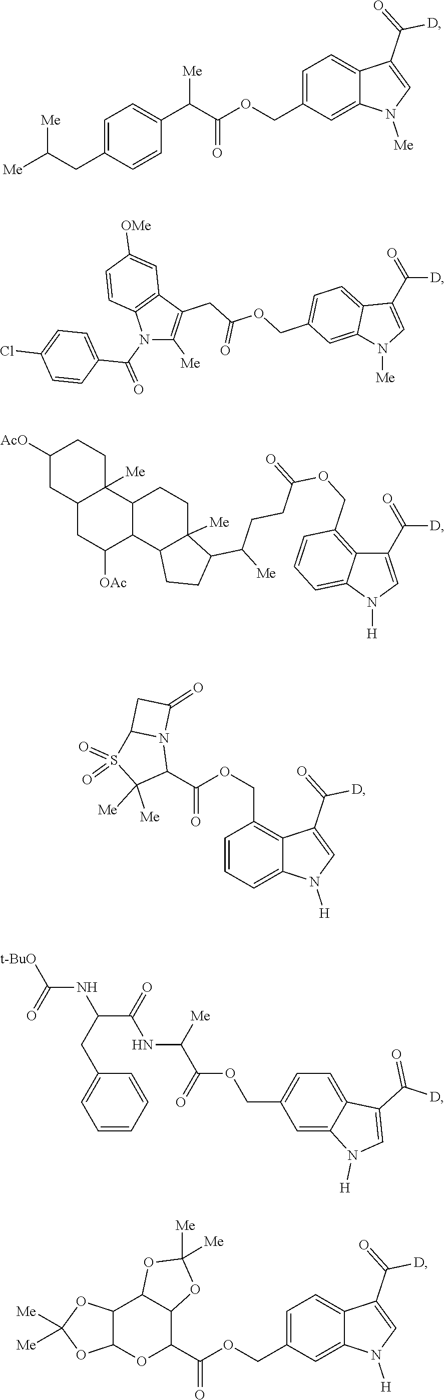

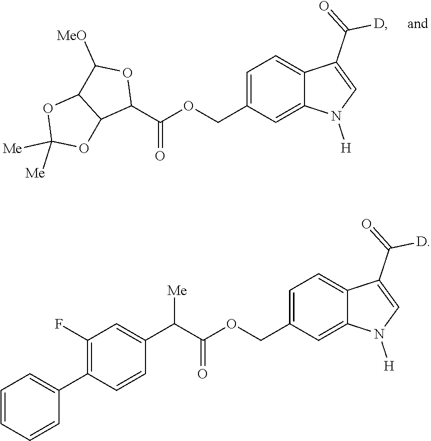

Clause 24. The compound of any one of clauses 15-23, or a salt thereof, wherein that compound has at least 50% deuterium incorporation at the deuterium label.

Clause 25. The compound of any one of clauses 15-24, or a salt thereof, wherein that compound has at least 75% deuterium incorporation at the deuterium label.

Clause 26. The compound of any one of clauses 15-25, or a salt thereof, wherein that compound has at least 90% deuterium incorporation at the deuterium label.

Clause 27. The compound of any one of clauses 15-26, or a salt thereof, wherein that compound has at least 95% deuterium incorporation at the deuterium label.

Clause 28. The compound of any one of clauses 15-27, or a salt thereof, wherein that compound has at least 99% deuterium incorporation at the deuterium label.

What is claimed is:

1. A method for preparing a deuterated compound of formula (I), or a salt thereof, 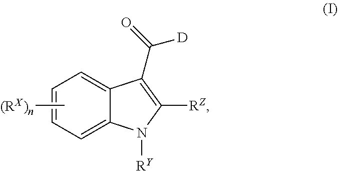
wherein:
R^{X} is halogen, C₁₋₄alkyl, C₁₋₂haloalkyl, R^{a}, —NO₂, —CN, —CO₂C₁₋₄alkyl, —C(O)C₁₋₄alkyl, —C(O)H, —OC₁₋₃alkylene-R^{a}, —C₁₋₃alkylene-R^{a}, -L¹-A¹, or -L²-A²;
R^{a} is C₃₋₄cycloalkyl, —OC₁₋₄alkyl, —OC₁₋₂haloalkyl, —OC₃₋₄cycloalkyl, or phenyl, wherein, at each occurrence, the phenyl is optionally substituted with 1-2 substituents selected from the group consisting of halogen, C₁₋₄alkyl, C₁₋₂haloalkyl, —NO₂, —CN, —OC₁₋₄alkyl, —OC₁₋₂haloalkyl, or —OC₃₋₄cycloalkyl;
R^{Y} is hydrogen, C₁₋₄alkyl, or an amine protecting group;
R^{Z} is hydrogen, C₁₋₄alkyl, C₂₋₄alkenyl, or unsubstituted phenyl;
L¹ is —C₁₋₃alkylene- or —OC₁₋₃alkylene-;
A¹ is a drug moiety;
wherein A¹ is linked to L¹ by 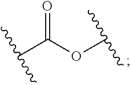 [SMILES: *OC(*)=O]
L² is —NH—;
A² is a drug moiety;
wherein A² is linked to L² by: 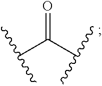 [SMILES: *C(*)=O]
and
n is 0, 1, or 2;
the method comprising:
mixing D-CO₂D with a compound of formula (II), 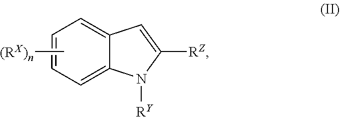
in an organic solvent to form a mixture; and
exposing the mixture of (i) to light, thereby producing the deuterated compound of formula (I), or a salt thereof.

2. The method of claim 1, wherein n is 0 or 1.

3. The method of claim 1, wherein R^{X} is halogen, C₁₋₄alkyl, —NO₂, —CN, —OC₁₋₄alkyl, —CO₂C₁₋₄alkyl, —C(O)H, -L¹-A¹, or -L²-A².

4. The method of claim 1, wherein L¹ is —CH₂—.

5. The method of claim 1, wherein the drug moiety at A¹ or A² is a non-steroidal anti-inflammatory drug (NSAID) moiety or a β-lactamase inhibitor moiety.

6. The method of claim 1, wherein R^{X} is selected from the group consisting of:

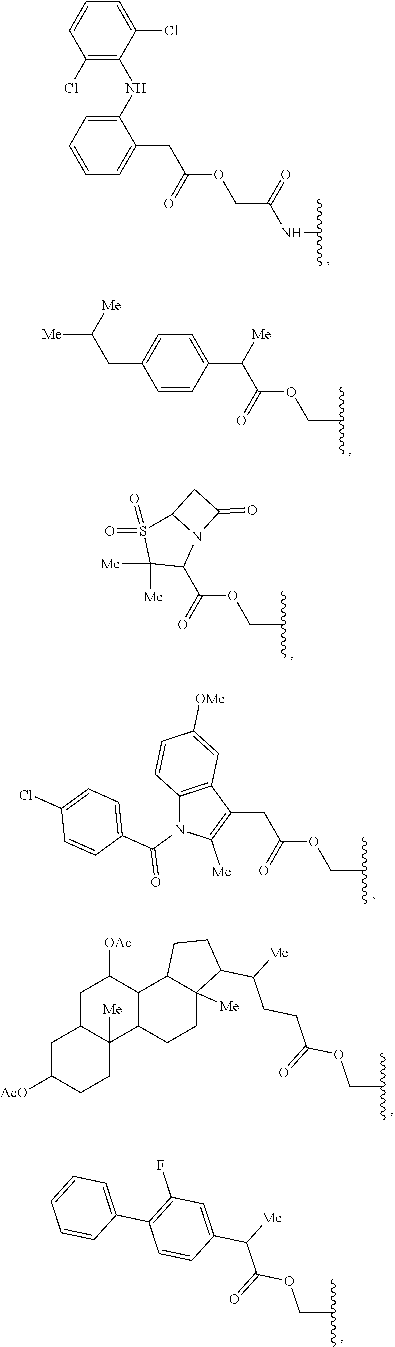

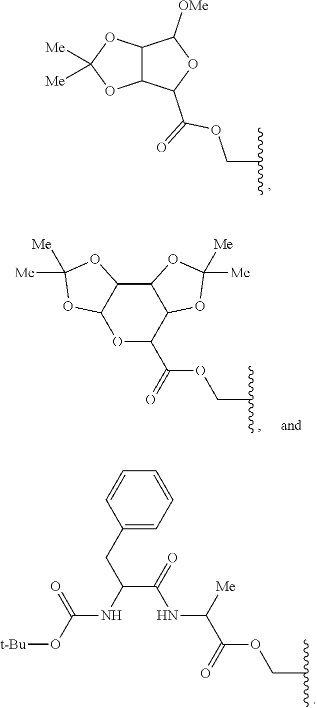 [SMILES: *COC(=O)C1OC(OC)C2OC(C)(C)OC12 ; *COC(=O)C1OC2OC(C)(C)OC2C2OC(C)(C)OC12 ; *COC(=O)C(C)NC(=O)C(Cc1ccccc1)NC(=O)OC(C)(C)C]

7. The method of claim 1, wherein R^{Y} is hydrogen, C₁₋₄alkyl,

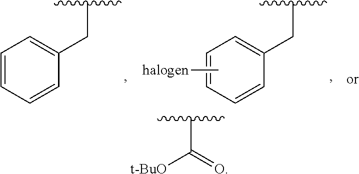

8. The method of claim 1, wherein R^{Z} is hydrogen, methyl,

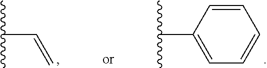

9. The method of claim 1, wherein the compound of formula (I) is selected from the group consisting of:

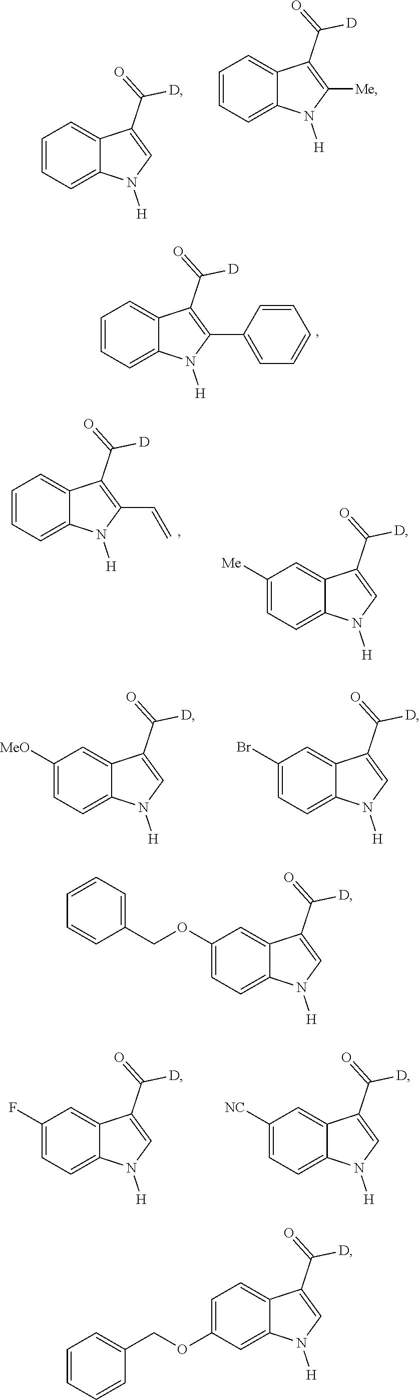

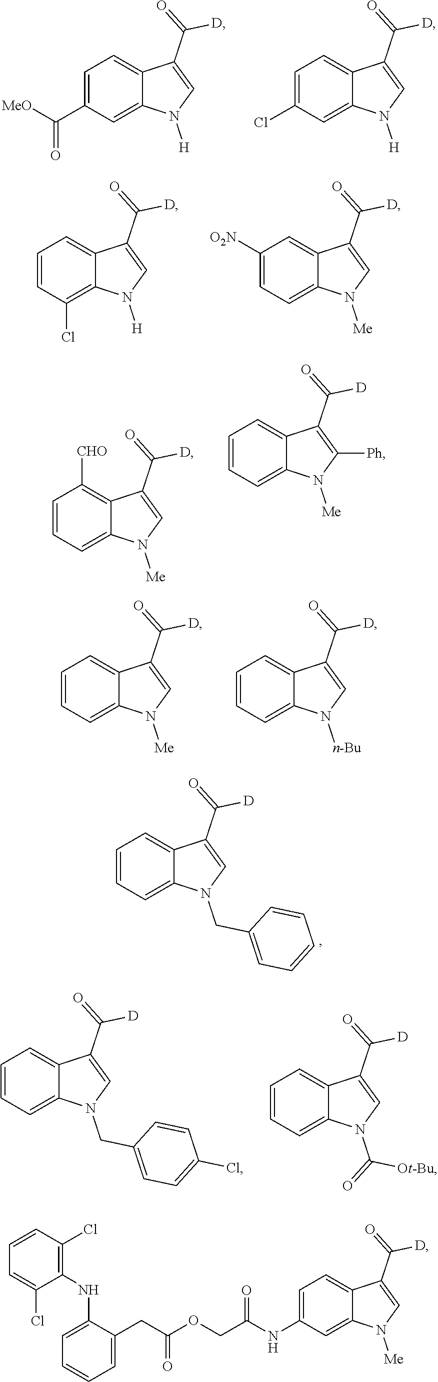

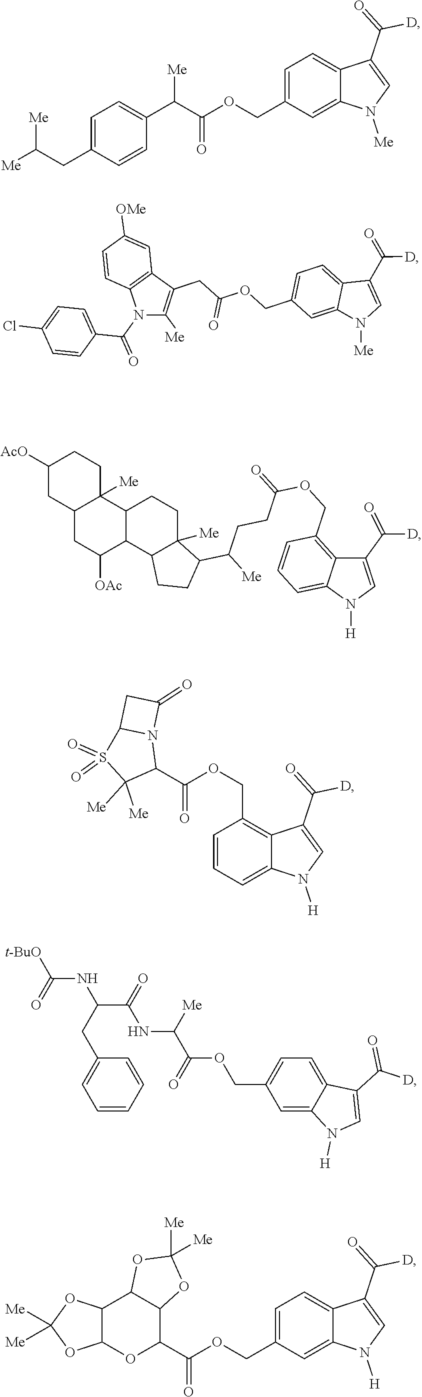

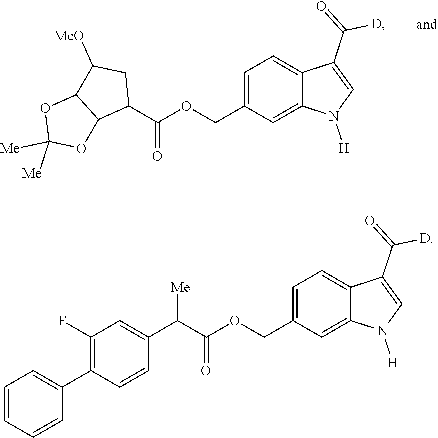

10. The method of claim 1, wherein the exposing the mixture of (i) to light occurs in the presence of air.

11. The method of claim 1, wherein the mixture is essentially water (H₂O) free.

12. The method of claim 1, wherein the base is sodium acetate (NaOAc).

13. The method of claim 1, wherein the mixture is catalyst free.

14. The method of claim 1, wherein the solvent is a polar aprotic solvent.

15. A deuterated compound of formula (I), or a salt thereof, 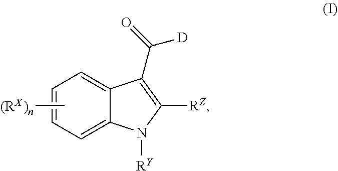
wherein:
Rₓ is halogen, C₁₋₄alkyl, C₁₋₂haloalkyl, R^{a}, —NO₂, —CN, —CO₂C₁₋₄alkyl, —C(O)C₁₋₄alkyl, —C(O)H, —OC₁₋₃alkylene-R^{a}, —C₁₋₃alkylene-R^{a}, -L¹-A¹, or -L²-A²;
R^{a} is C₃₋₄cycloalkyl, —OC₁₋₄alkyl, —OC₁₋₂haloalkyl, —OC₃₋₄cycloalkyl, or phenyl, wherein, at each occurrence, the phenyl is optionally substituted with 1-2 substituents selected from the group consisting of halogen, C₁₋₄alkyl, C₁₋₂haloalkyl, —NO₂, —CN, —OC₁₋₄alkyl, —OC₁₋₂haloalkyl, or —OC₃₋₄cycloalkyl;
R^{Y} is hydrogen, C₁₋₄alkyl, or an amine protecting group;
R^{Z} is hydrogen, C₁₋₄alkyl, C₂₋₄alkenyl, or unsubstituted phenyl;
L¹ is —C₁₋₃alkylene- or —OC₁₋₃alkylene-;
A¹ is a drug moiety;
wherein A¹ is linked to L¹ by 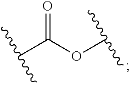 [SMILES: *OC(*)=O]
L² is —NH—;
A² is a drug moiety;
wherein A² is linked to L² by: 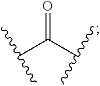 [SMILES: *C(*)=O]
and
n is 0, 1, or 2.

16. The compound of claim 15, or a salt thereof, wherein n is 0 or 1.

17. The compound of claim 15, or a salt thereof, wherein R^{X} is halogen, C₁₋₄alkyl, —NO₂, —CN, —OC₁₋₄alkyl, —CO₂C₁₋₄alkyl, —C(O)H, -L¹-A¹, or -L²-A².

18. The compound of claim 15, or a salt thereof, wherein L¹ is —CH₂—.

19. The compound of claim 15, or a salt thereof, wherein the drug moiety at A¹ or A² is a non-steroidal anti-inflammatory drug (NSAID) moiety or a β-lactamase inhibitor moiety.

20. The compound of claim 15, or a salt thereof, wherein R^{X} is selected from the group consisting of

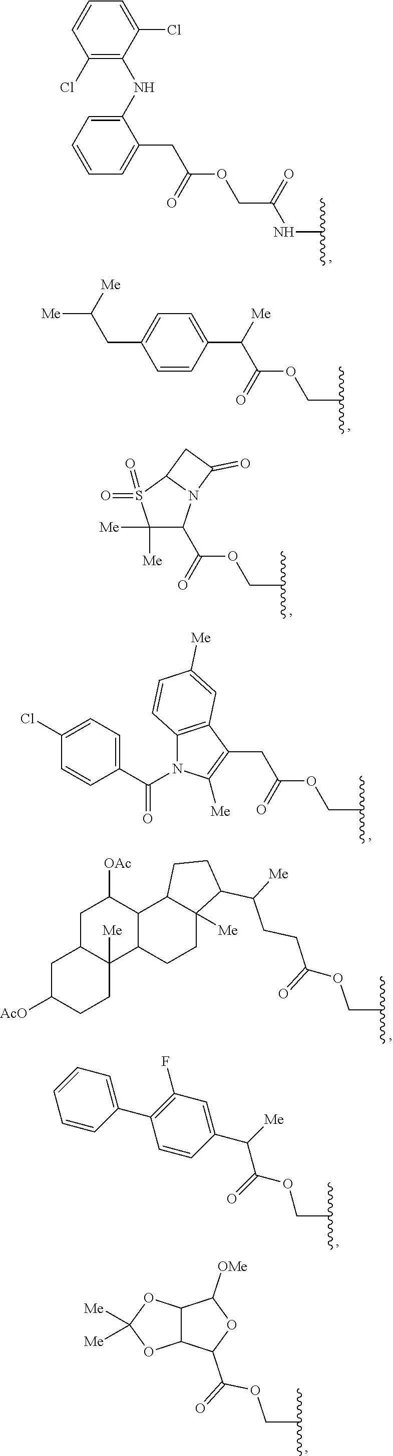

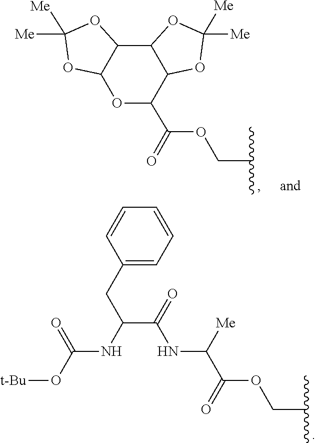

21. The compound of claim 15, or a salt thereof, wherein R is hydrogen, C₁₋₄alkyl,

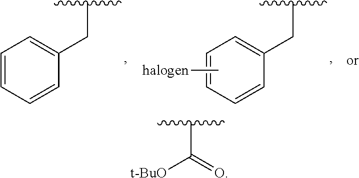

22. The compound of claim 15, or a salt thereof, wherein R^{Z} is hydrogen, methyl,

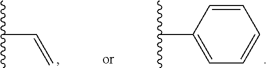

23. The compound of claim 15, selected from the group consisting of:

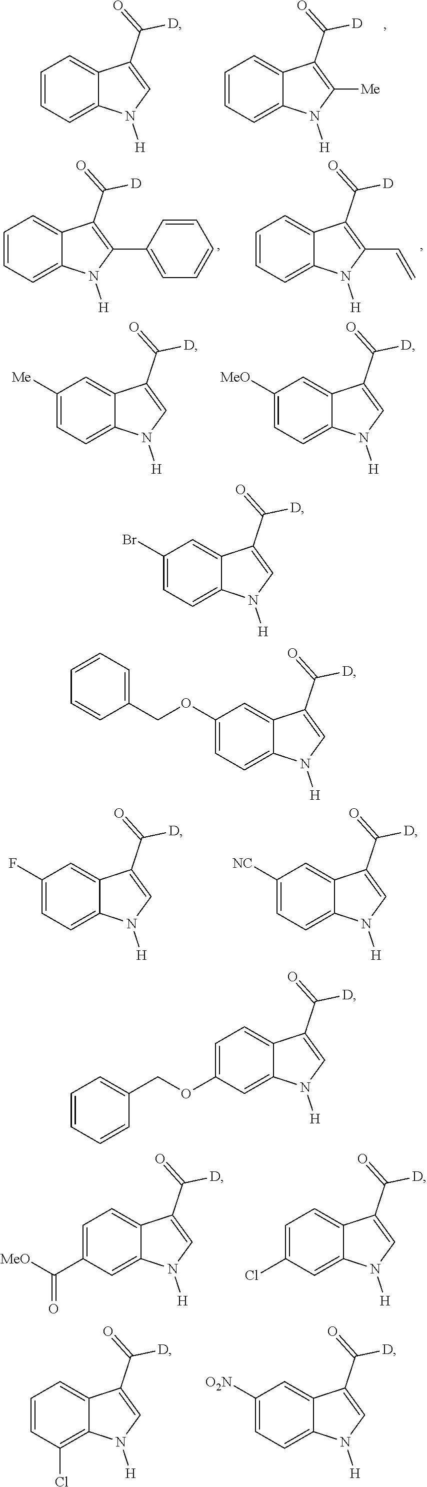

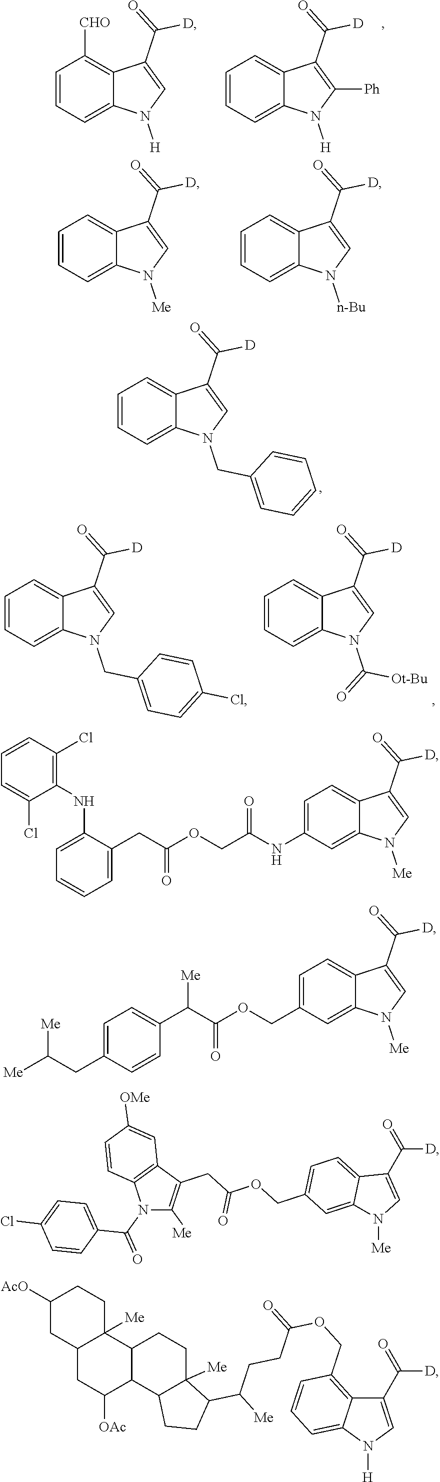

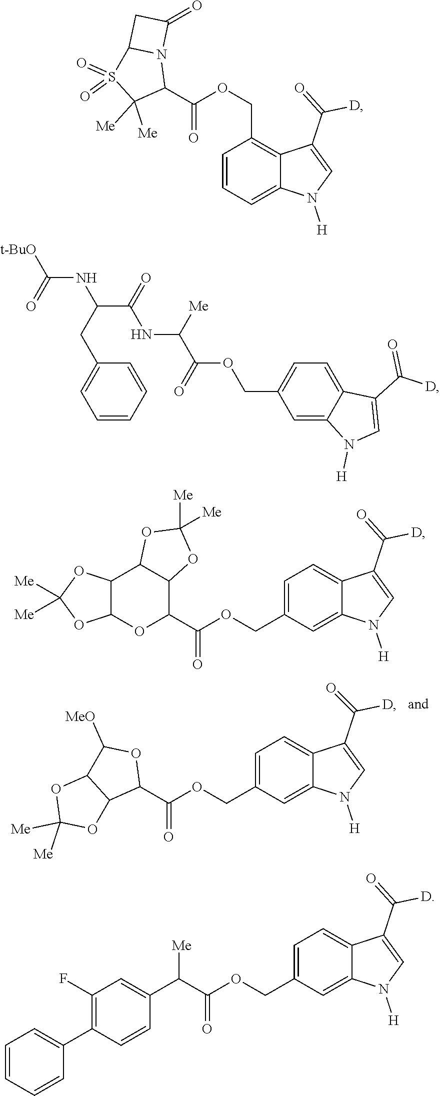

24. The compound of claim 15, or a salt thereof, wherein that compound has at least 50% deuterium incorporation at the deuterium label.

25. The compound of claim 15, or a salt thereof, wherein that compound has at least 75% deuterium incorporation at the deuterium label.

26. The compound of claim 15, or a salt thereof, wherein that compound has at least 90% deuterium incorporation at the deuterium label.

27. The compound of claim 15, or a salt thereof, wherein that compound has at least 95% deuterium incorporation at the deuterium label.

28. The compound of claim 15, or a salt thereof, wherein that compound has at least 99% deuterium incorporation at the deuterium label.
